(19) United States
(12) Patent Application Publication
(10) Pub. No.: US 2026/0058015 A1
(43) Pub. Date: Feb. 26, 2026

(54) METHOD AND SYSTEM OF PREDICTING A CLINICAL OUTCOME OR CHARACTERISTIC

(71) Applicant: BenevolentAI Technology Limited, London (GB)

(72) Inventors: Harry ROSE, London (GB); Anna Muñoz FARRÉ, London (GB); Dilini KOTHALAWALA, London (GB); Antonios Poulakakis DAKTYLIDIS, London (GB); Andrea Rodriguez MARTINEZ, London (GB)

(73) Assignee: BenevolentAI Technology Limited, London, LN (GB)

(21) Appl. No.: 19/106,040

(22) PCT Filed: Aug. 24, 2023
(86) PCT No.: PCT/EP2023/073236

§ 371 Date: Feb. 24, 2025

(30) Foreign Application Priority Data
Aug. 25, 2022 (GB) 2212399.6
May 30, 2023 (EP) 23176154.5

(51) Int. Cl. G16H 50/20; G16H 10/40; G16H 10/60; G16H 50/70

(52) U.S. Cl. CPC G16H 50/20; G16H 10/40; G16H 10/60; G16H 50/70

(57) ABSTRACT

A computer-implemented method of training a machine learning model to predict a clinical outcome or characteristic based on a patient's clinical history is disclosed. The method comprises: providing training data comprising structured electronic health record data for a plurality of patients, the structured electronic health record data comprising a plurality of clinical observations, each clinical observation having a text description and an associated time stamp, wherein the training data for each patient is labelled with one or more labels, each representing a clinical outcome or characteristic; converting each patient's electronic health record data into a text sequence comprising the text descriptions concatenated in sequence of the time stamps; inputting the text sequence into a machine learning model; and training the machine learning model to predict a clinical outcome or characteristic based on the input text sequence.

### FIELD OF THE INVENTION

The present invention relates to a method and system for training a machine learning model to predict a clinical outcome or characteristic based on electronic health record data. The present invention also relates to a method and system of using a trained machine learning model to predict a clinical outcome or characteristic by processing health record data.

### BACKGROUND

Electronic health records (EHRs) describe the information on patients' health acquired during the day-to-day utilisation of the healthcare system. These include clinical covariates and phenotypes, laboratory tests, primary and secondary care records, information from disease databases, free text, clinical images and, increasingly, genomic data. EHRs collate a patient's medical history over time and, ideally, include all key administrative and clinical data relating to a patient's care under a particular provider, where different providers such as primary care providers, hospitals, laboratory test centres and pharmacies will maintain their own digital record for a patient. These longitudinal data sets often span decades and recreate the patients' medical history ‘from cradle to grave’.

The wealth of information and the longitudinal nature of the data raises the question of whether electronic health care records can be utilised using data modelling and computation techniques to make predictions about a patient's health. For example, there is the question of whether a patient's health care record can be used to predict a missing diagnosis, i.e. a diagnosis that is not present in the electronic health record but may be predicted from the clinical observations that are present. Similarly, it may be that the data can be used to predict a risk factor of developing a particular health condition in future. Since the electronic health records are maintained as structured databases, there have been attempts to leverage the structured data using machine learning techniques to make health condition predictions.

However, there are a number of technical challenges with this approach. Firstly, to understand patient trajectories fully, it is necessary to combine multiple structured electronic health record data sources together, for example primary and secondary care records. However, different providers use different data structures with differing ontologies to describe the clinical data. In particular, different data providers use their own system of clinical codes which correspond to different clinical observations, measurements or tasks. The differing data structures present a significant technical challenge to combine the multiple modalities in a single model to make predictions. Current approaches generally rely on manually curated mappings between ontologies and are often prone to error and can lose information and the granularity of the original data during mapping. These existing techniques add noise and bias to already existing sources of noise, error and missing values in EHRs.

Additionally, existing techniques are prone to overfitting to prevalent diseases, particularly with respect to patients having comorbidities.

For these reasons there exists a need for a new technique for leveraging electronic health care records to make predictions regarding a patient's health condition, which makes progress in addressing the above problems. In particular, there is a need for a method that can combine different types of electronic health records in order to make improved predictions.

Additionally, it is a further object of the invention to provide a tool which allows EHR data to be used for interpreting disease progression patterns and for stratifying patients into clinically-relevant subgroups with different aetiological and prognostic profiles. This is difficult to achieve using EHR data which is collected from various different sources, and existing methods are known to suffer from poor clinical interpretability. An advancement in this regard would enable enhanced medical decision making and facilitate the provision of improved treatment plans for patients.

### SUMMARY OF INVENTION

In a first aspect of the invention there is provided a computer-implemented method of training a machine learning model to predict a disease diagnosis based on a patient's clinical history, the method comprising: providing training data comprising structured electronic health record data for a plurality of patients, the structured electronic health record data comprising a plurality of clinical observations, each clinical observation having a text description and an associated time stamp, wherein the training data for each patient is labelled with one or more labels, each representing a disease diagnosis; converting each patient's electronic health record data into a text sequence comprising the text descriptions concatenated in sequence of the time stamps; inputting the text sequence into a machine learning model and training the machine learning model to predict a disease diagnosis based on the input text sequence.

Generally electronic health records of different types use different ontologies comprising different clinical codes to describe clinical observations. Different health record types, for example primary and secondary care records, may use different codes to describe the same diagnosis or observation. They may also define health conditions at different granularities. However, these different health record types all include a text description of each code. The present method utilises this by using text as the input into the model. By representing a patient's clinical history as a sequence of text comprising the text descriptions of the clinical codes across their health records, it is possible to combine different data types into a single input for training a machine learning model. Furthermore, combining clinical observations into a text sequence allows temporal information to be encoded into the input for a machine learning model. The present invention improves on the prior art which requires lossy mapping techniques to combine different data types and instead may include the full digital health record, minimising losses and improving predictions. In this way, pre-training language models can be harnessed to learn rich representations of a patient's EHR which can then be used to predict a missing diagnosis or risk of developing a disease. The method can also be applied for disease clustering and for performing genome wide association studies.

A “clinical outcome or characteristic” preferably refers to a clinical outcome or characteristic of the patient. A characteristic may preferably comprise a health condition, wherein the training data is labelled with one or more labels, each indicating whether the patient has a particular health condition. The machine learning model is then trained to predict whether the patient has each health condition based on the input text sequence.

The structured electronic health record data preferably comprises a record of a patient's interaction with a health care service. It preferably comprises a sequence of events such as clinical observations. The structured electronic health record data may comprise one or more of: clinical covariates and phenotypes, laboratory tests, primary and secondary care records, information from disease databases, free text, clinical images and genomic data.

The structured electronic health care record data comprises an ontology comprising a plurality of clinical codes, each indicating a particular clinical observation. Each clinical code may be associated with a text description, describing the clinical observation that it indicates, for example a disease diagnosis, a treatment, a measurement of a biomarker, a laboratory text result or a clinical procedure performed on the patient. The text description may be part of the structured electronic health record data or a database comprising the text descriptions associated with each clinical code may be stored elsewhere. In these examples the method may comprise accessing the database to determine the text description associated with each clinical code in a patient's electronic health care record data.

The time stamp may comprise a date and or time at which the clinical observation of the patient was made or, alternatively or additionally, it may comprise the age of the patient when the clinical observation of the patient was made. The labels are preferably binary labels indicating true/false or present/not present (i.e. in the form of “1” or “0”) specifying whether the particular clinical outcome or clinical characteristic is relevant to that patient.

Preferably, the step of converting each patient's electronic health record data into a text sequence comprises: for patients labelled with a positive clinical outcome or characteristic, masking one or more words associated with the clinical outcome or characteristic from the text sequence before inputting to the machine learning model. In this way, the model is trained to learn to predict a clinical outcome or condition, such as a disease diagnosis, based on the patient's clinical history (without relying on the words that are directly associated with the clinical outcome and condition). The trained model can then be applied at prediction time to predict a missing or unknown clinical outcome or characteristic for a patient based on their electronic health care record. Here the term “masking” can comprise removing one or more words from the text sequence. It can equally comprise replacing the words with a mask, for example where the text is represented by a sequence of text tokens, replacing the text tokens representing the one or more words with a mask token.

Preferably, the method comprises: masking a first percentage of words associated with the clinical outcome or characteristic from the text sequence; randomly replacing a second percentage of words associated with the clinical outcome or characteristic from the text sequence; and keeping a third percentage of words associated with the clinical outcome or characteristic from the text sequence. In this way, noise may be introduced during training, thereby increasing the robustness of the model.

Preferably, when a patient's electronic health record data is labelled with multiple positive clinical outcome or characteristic labels, the method comprises: generating a duplicate text sequence for each positive clinical outcome or characteristic label; applying the steps of claim 2 or claim 3 for each duplicate text sequence to remove words associated with the corresponding positive clinical outcome or characteristic. There is a particular technical challenge associated with the problem of training a model for predicting a clinical outcome or characteristic in the presence of comorbidities, for example when a patient has a positive label for two or more, possibly related, diseases. This data augmentation method addresses this problem.

Preferably, the method further comprises: computing, for each duplicate text sequence, loss weights for use in a loss function against which the machine learning model is trained, wherein, for each respective duplicate text sequence, words that are associated with a positive labelled clinical outcome or characteristic that are not masked in the respect duplicate text sequence are assigned a loss weight of 0. In this way, it is possible to avoid overfitting to prevalent clinical outcomes or characteristics when a text sequence contains descriptions that are strongly associated with multiple positive clinical outcomes or characteristic labels. In one example, the loss function may be a mean-reduced binary cross-entropy loss function.

Preferably, the structured electronic health record data comprises a plurality of different electronic health record data types, each having a different ontology with different clinical codes representing the clinical observations, each clinical code having a text description, the method comprising: combining the text descriptions from each data type into the text sequence in the order of their associated time stamp.

Preferably, wherein the electronic health record data types comprise one or more of: a primary care health record, a secondary care health record such as a hospital health record, a biomarker health record, a medication history record.

Preferably, wherein training the machine learning model comprises a fine-tuning step and a classification training step, the fine-tuning step comprising: masking one or more words from the text sequence, inputting the masked text sequence into the machine learning model and training the machine learning model to predict the masked words, the classification training step comprising: inputting the text sequence into a machine learning model and training the machine learning model to predict the clinical outcome or characteristic based on the input text sequence. The fine-tuning step based on masked language modelling trains the model to learn representations which encode the semantics of the text sequences comprising clinical observation descriptions. The classification training step further refines the representations learned by the model to make them usable for classification to predict a clinical outcome or characteristic. Preferably the machine learning model comprises an encoder and the fine-tuning step comprises training the encoder using the masking objective. The classification training step preferably comprises adding a classification layer (e.g. a fully connected linear layer) and training the encoder and classification layer together.

Preferably, the method further comprises: encoding the text sequences by mapping an input representation of each text sequence to an output representation and training the machine learning model to predict the clinical outcome or characteristic based on the output representation.

Preferably, the method comprises: performing tokenisation on the text sequence to form a sequence of word-piece tokens representing the text sequence, and inserting the sequence of word-piece tokens into the model. Each word piece token preferably comprises a word or sub-word portion of text. The sequence of word-piece tokens are preferably mapped to embeddings at the input layer of the encoder.

Preferably, the training data is labelled with a plurality of binary labels, each representing whether the patient has a clinical outcome or characteristic, wherein the machine learning model is trained to predict the existence of the clinical outcome or characteristic.

Preferably, the labelling of the training data is carried out automatically using a clinical outcome or characteristic definition algorithm configured to assign a clinical outcome or characteristic based on one or more clinical codes present in a patient's electronic health record data.

Preferably, each clinical observation in the structured electronic health record data further comprises one or more continuous measurements, where the method further comprises inputting the one or more continuous measurements together with the corresponding text descriptions into the model, and training the machine learning model to predict a clinical outcome or characteristic based on the input text sequence and the one or more continuous measurements.

Preferably, the one or more continuous measurements comprise at least one of: age of the patient, time of the clinical observation, and position of the patient.

Preferably, the method further comprises encoding the input text sequence into text embeddings, encoding the one or more continuous features into continuous feature embeddings, concatenating the text embeddings and continuous feature embeddings into an input representation, and training the machine learning model to predict the clinical outcome or characteristic based on the concatenated input representation of the text embeddings and continuous feature embeddings. The skilled person will understand that, in this context, the term “encoding” refers to mapping the input text sequence or continuous features to respective embeddings for use by the encoder. In further examples, the text embeddings may additionally or alternatively be concatenated with positional embeddings which encode the position of the corresponding word or word piece token in the input text sequence.

Preferably, wherein the machine learning model comprises: an encoder for mapping the input text sequence to an output representation; and a classifier layer that receives the output representation and outputs a predicted clinical outcome or characteristic. The skilled person will understand that the classifier layer may also be generally referred to as a decoder.

Preferably, wherein the encoder comprises a Transformer encoder, a Long Short-Term Memory (LSTM) encoder, or a Gated Recurrent Unit (GRU) encoder.

Preferably, wherein the encoder comprises a pre-trained language model, pre-trained using masked language modelling on biomedical literature data.

Preferably, wherein the pre-trained language model is further pre-trained using masked language modelling on text sequences formed by concatenating the text descriptions of electronic health record data.

Preferably, wherein the classifier layer is trained to output prediction of a clinical outcome or characteristic, where the prediction comprises a probability of the patient having that clinical outcome or characteristic.

According to a second aspect of the invention, there is provided a computer-implemented method of predicting a clinical outcome or characteristic based on a patient's clinical history, the method comprising: obtaining structured electronic health record data for the patient, the structured electronic health record data comprising a plurality of clinical observations, each clinical observation having a text description and an associated time stamp; converting the patient's electronic health record data into a text sequence by concatenating the text descriptions in sequence of the time stamps; inputting the text sequence into a machine learning model trained to predict a clinical outcome or characteristic based on the input text sequence; and outputting the clinical outcome or characteristic.

Preferably, the machine learning model is trained using the method of the first aspect. The model may have any of the features described above under the first aspect.

Preferably, the machine learning model is trained to provide a prediction for a plurality of clinical outcomes or characteristics, the method comprising outputting the plurality of clinical outcomes or characteristics.

Preferably, the machine learning model is configured to provide a probability of the patient having the clinical outcome or characteristic for each of the plurality of clinical outcomes or characteristics.

Preferably, the clinical outcome or characteristic comprises: a phenotype, a disease diagnosis, a medical condition, a clinical outcome, a medical event, or a medical state.

Preferably, the machine learning model comprises an encoder, wherein the encoder has been trained to map the text sequences to embeddings which encode the semantics of the text sequences; wherein the method comprises performing dimensionality reduction on each set of embeddings to transform each set of embeddings into a respective reduced dimensionality embedding. Preferably, the clinical outcome or characteristic comprises a disease diagnosis.

Preferably, the clinical observations comprise disease diagnoses. Preferably clinical observations other than disease diagnoses have been removed from the input data.

Preferably, wherein the method further comprises: computing measures of association between the reduced dimensionality embeddings and clinical factors derived from the structured electronic health record data of the plurality of patients, the clinical factors preferably comprising one or more of symptoms, laboratory tests, vital signs, medication, and medical conditions co occurring with the particular disease.

Preferably, the method comprises determining a patient group or disease subtype based on the computed measures of association.

Preferably, the method comprises performing clustering on the reduced dimensionality embeddings. Preferably the method comprises determining a patient group or disease subtype based on the clustered reduced dimensionality embeddings. Preferably the method comprises determining a treatment plan based on the patient group or disease subtype. Preferably the method comprises performing genetic analysis on patients determined as falling within the patient groups or disease subtypes. Preferably the method comprises determining a drug compound or treatment plan based on the genetic analysis.

Preferably, wherein the measures of association comprises point biserial coefficients.

Preferably, wherein the reduced dimensionality embeddings are two-dimensional vectors each comprising a first component and a second component.

Preferably, wherein computing the measures of association between the reduced dimensionality embeddings and the clinical factors derived from the structured electronic health record data of the plurality of patients comprises: for each clinical factor, calculating a first point biserial coefficient between the clinical factor and the first components of the two-dimensional vectors; and for each clinical factor, calculating a second point biserial coefficient between the clinical factor and the second components of the two-dimensional vectors,

Preferably, wherein the measures of association comprises the first and second point biserial coefficients. Preferably the method further comprises: for each clinical factor, calculating a Euclidean norm based on the corresponding first point biserial coefficient and second point biserial coefficient.

According to a third aspect of the invention, there is provided a computer-implemented method, comprising: obtaining structured electronic health record data for a plurality of patients which have all received a diagnosis for a particular disease, wherein the structured electronic health record data for each patient comprises a plurality of clinical observations, each clinical observation having a text description and an associated time stamp; dividing each patient's structured electronic health record data into a plurality of datasets, wherein each dataset comprises a sequential set of clinical observations; converting each dataset into a respective text sequence by concatenating the text descriptions of each dataset in sequence of the time stamps; inputting each text sequence into an encoder of a machine learning model, wherein the encoder has been trained to map the text sequences to embeddings which encode the semantics of the text sequences; mapping each text sequence to a respective set of embeddings using the encoder; and performing dimensionality reduction on each set of embeddings to transform each set of embeddings into a respective reduced dimensionality embedding.

In this way, an embedding space is provided which captures and represents complex disease stages or themes within a patient's medical history. The reduced dimensionality embeddings therefore provide clinically meaningful insight which can be utilised to enhance medical decision making and to provide improved treatment plans for patients.

Preferably, the machine learning model has been trained to predict a clinical outcome or characteristic based on the input text sequence.

Preferably, the machine learning model is trained using the method of the first aspect. The model may have any of the features described above under the first aspect.

Preferably, the method further comprises evaluating progression patterns of the particular disease based on the reduced dimensionality embeddings.

Preferably, wherein each of the plurality of datasets do not include clinical observations associated with the particular disease.

Preferably, wherein each of the plurality of datasets consist of clinical observations corresponding to diseases diagnoses.

Preferably, wherein the method further comprises: performing tokenisation on each text sequence to form a sequence of word-piece tokens representing the text sequence; and inserting each sequence of word-piece tokens into the encoder.

Preferably, wherein the method further comprises: computing measures of association between the reduced dimensionality embeddings and clinical factors derived from the structured electronic health record data of the plurality of patients.

Preferably, wherein the measures of association comprises point biserial coefficients.

Preferably, wherein the reduced dimensionality embeddings are two-dimensional vectors each comprising a first component and a second component.

Preferably, wherein computing the measures of association between the reduced dimensionality embeddings and the clinical factors derived from the structured electronic health record data of the plurality of patients comprises: for each clinical factor, calculating a first point biserial coefficient between the clinical factor and the first components of the two-dimensional vectors; and for each clinical factor, calculating a second point biserial coefficient between the clinical factor and the second components of the two-dimensional vectors, Preferably, wherein the measures of association comprises the first and second point biserial coefficients.

Preferably, wherein the method further comprises: for each clinical factor, calculating a Euclidean norm based on the corresponding first point biserial coefficient and second point biserial coefficient, Preferably, wherein the measures of association comprises the Euclidean norms.

Preferably, wherein the clinical factors derived from the structured electronic health record data of the plurality of patients comprise one or more of: symptoms, laboratory tests, vital signs, medication, and medical conditions co occurring with the particular disease.

Preferably, wherein each dataset for each patient is associated with a respective time period defined with respect to the patient's date of diagnosis for the particular disease, and wherein the method further comprises: performing linear interpolation on the reduced dimensionality embeddings to generate interpolated reduced dimensionality embeddings which are temporally aligned between patients; performing time series clustering on the interpolated reduced dimensionality embeddings to identify a plurality of patient subtypes.

Preferably, the method further comprises determining a treatment plan based on the patient subtype(s). Preferably the method further comprises performing genetic analysis on patients determined as falling within the patient subtypes.

Preferably the method comprises determining a drug compound or treatment plan based on the genetic analysis.

According to a fourth aspect, there is provided a computer program product comprising instructions which, when the program is executed by a computer, cause the computer to carry out the method of any of the first to third aspects.

According to a fifth aspect of the invention, there is provided a system comprising a processor configured to perform the method of any of the first to third aspects.

### BRIEF DESCRIPTION OF DRAWINGS

FIG. 1 is a flowchart showing method steps for training a machine learning model to predict a clinical outcome or characteristic based on a patient's clinical history;

FIG. 2 is a schematic diagram illustrating different types of electronic health record data being fused into a single text sequence;

FIG. 3 is a schematic diagram illustrating the mapping of a unified clinical history to an input text sequence with a multi-label target vector of aggregated phenotype labels from an oracle annotation;

FIG. 4 is an example illustration of a text sequence being masked before being input to a machine learning model for fine-tuning;

FIG. 5 is an example illustration of a masking strategy for a given patient history with heart failure, in which words associated with heart failure are either deleted, swapped or kept in the text sequence before the text sequence is provided to a machine learning model for classification training;

FIG. 6 is a example illustration of a text input sequence being duplicated and assigned masking and loss weights vectors;

FIG. 7 is a flowchart showing method steps for predicting a clinical outcome or characteristic based on a patient's clinical history;

FIG. 8 is a schematic diagram showing an exemplary model architecture;

FIG. 9 is series of AUC-ROC curves for true positives in a test set;

FIG. 10 is a pair of graphs showing predicted probabilities for Type II Diabetes Mellitus;

FIG. 11 is graph showing HbA1c distribution across patient groups;

FIG. 12 is a graph showing T2DM polygenic risk score vs T2DM probability prediction;

FIG. 13 is a pair of graphs showing distributions of number of GP and hospital codes per patient group and number of hospital codes per patient group respectively;

FIG. 14 is a graph showing death survival plots for different patient groups; and

FIG. 15 is a graph showing Framingham and QRISK cardiovascular scores distribution across different patient groups;

FIG. 16 is a flowchart showing method steps for generating dimensionality reduced embeddings;

FIG. 17 is a flowchart showing method steps for computing measures of association between the reduced embeddings and clinical factors;

FIG. 18 is flowchart showing method steps for identifying patient subtypes;

FIG. 19 is schematic diagram illustrating an example of dividing EHR data from a patient into a plurality of snapshots;

FIG. 20 is a schematic flow diagram illustrating the application of an exemplary model architecture;

FIG. 21 is a schematic diagram illustrating the computation of correlation between the reduced embeddings and clinical factors for each snapshot;

FIG. 22 is a pair of graphs illustrating patient clustering on trajectories (with an example 5 year time step) on simulated data;

FIGS. 23A and 23B are graphs illustrating exemplary associations between reduced embeddings and clinical factors;

FIG. 24 is an exemplary UMAP visualization of 4 clusters (mean per cluster and time window); and

FIG. 25 is a number of exemplary graphs illustrating disease theme prevalence for each cluster and snapshot.

### DETAILED DESCRIPTION

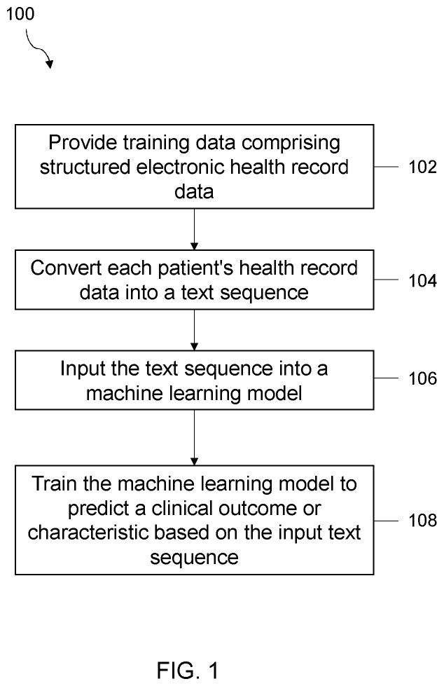

FIG. 1 illustrates a method 100 of training a machine learning model to predict a clinical outcome or characteristic based on a patient's clinical history. As used herein, the term “clinical outcome” will be understood to encompass any medical event or outcome associated with a patient, such as the occurrence of a heart attack, death or survival, or the need for dialysis. The term “clinical characteristic” will be understood to encompass any medical condition, disease diagnosis, phenotype, observable trait or characteristic associated with a patient. The term does not necessarily refer to one disease, for example combinations of attributes may define a disease subtype (e.g. different COPD phenotypes). The term also encompasses differences between patient groups, i.e. it may not refer to a commonly defined disease, but instead may refer to a specific patient group within a disease, such as obese type 2 diabetes patients.

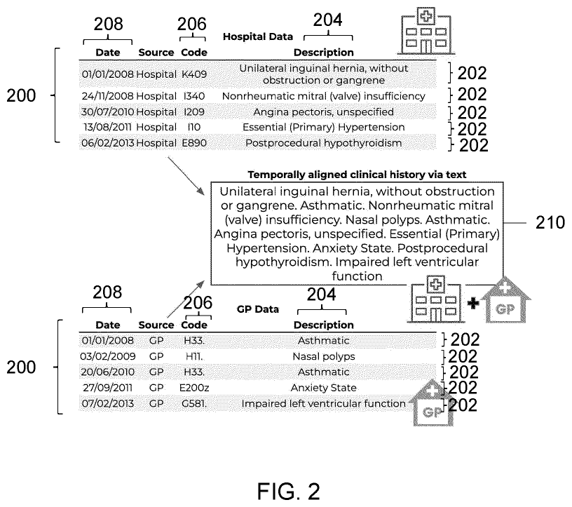

The method begins at step 102, wherein training data comprising structured electronic health record data 200 for a plurality of patients is provided. The term “electronic health record data” will be understood to encompass one or more types of health data, including primary healthcare records (e.g. GP data), secondary healthcare records (e.g. hospital data), biomarker health records, and/or medication history records. Exemplary electronic health record data 200 are illustrated in FIG. 2.

The electronic health record data 200 for each patent comprise a plurality of clinical observations 202 which are composed of diagnostic codes 206 having an associated description, i.e. a text description 204 of the diagnostic code 206 (also referred to as a textual descriptor). Examples of diagnostic codes 206 include ICD9/ICD10 codes and Read2/Read3 codes.

Each clinical observation 202 also includes an indication of the time at which the clinical observation 202 was taken, i.e. a time stamp 208 which is an example of a continuous measurement. One or more other continuous measurements may also be included in each clinical observation 202, such as the age of the patient at the clinical observation 202, and/or the position of the patient, e.g. geographical location.

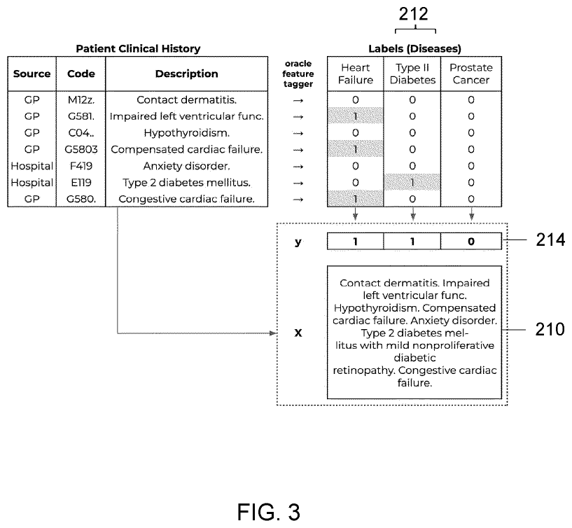

The training data is assigned one or more labels indicating whether each clinical observation 202 is associated with a clinical outcome or characteristic. The labels are assigned based on the text descriptions 204 and/or the diagnostic codes 206. For example, as illustrated in FIG. 3, the text description “impaired left ventricular function” and associated diagnostic code “G581” results in the corresponding clinical observation 202 being assigned a positive label for the clinical characteristic of heart failure. The text description “Type 2 diabetes mellitus” and associated diagnostic code “E119” results in the corresponding clinical observation 202 being assigned a positive label for the clinical characteristic of type II diabetes. These labels may be aggregated by mapping the labels to a multi-hot label vector 214 which is associated with the text sequence 210. Preferably, the labelling of the training data is carried out automatically using a clinical outcome or characteristic definition algorithm configured to assign a clinical outcome or characteristic based on the diagnostic code(s) 206 and/or text description 204 of the clinical observation 202. This process may be referred to as oracle feature tagging. An example of a clinical outcome or characteristic definition algorithm is the CALIBER phenotyping algorithm.

Although here the labels are determined via the clinical codes present in the electronic health records, in other examples the presence of the clinical outcome or characteristic may be determined separately and not via the data included in the patient clinical history used to train the model. For example a label indicating a particular disease diagnosis may be assigned based on a medication record for the patient, whether the medication record does not form part of the electronic health care record data used to train the model.

At step 104 each patient's electronic health record data 200 is converted into a text sequence 210 which is a concatenation of the text descriptions 204 ordered in time, i.e. in sequence of the time stamps 208. This approach allows different types of structured electronic health record data 200 to be combined without the loss of information or granularity which is associated with existing approaches that rely on manually curated mappings between ontologies and diagnostic codes. For example, as shown in FIG. 2, text descriptions 204 from a plurality of data sources (e.g. GP data and hospital data) each having a different ontology with different clinical codes 206 may be combined into the text sequence 210 in the order of their associated timestamps 208.

At step 106, the text sequence 210 is input into a machine learning model and, at step 108, the machine learning model is trained to predict a clinical outcome or characteristic based on the input text sequence 210.

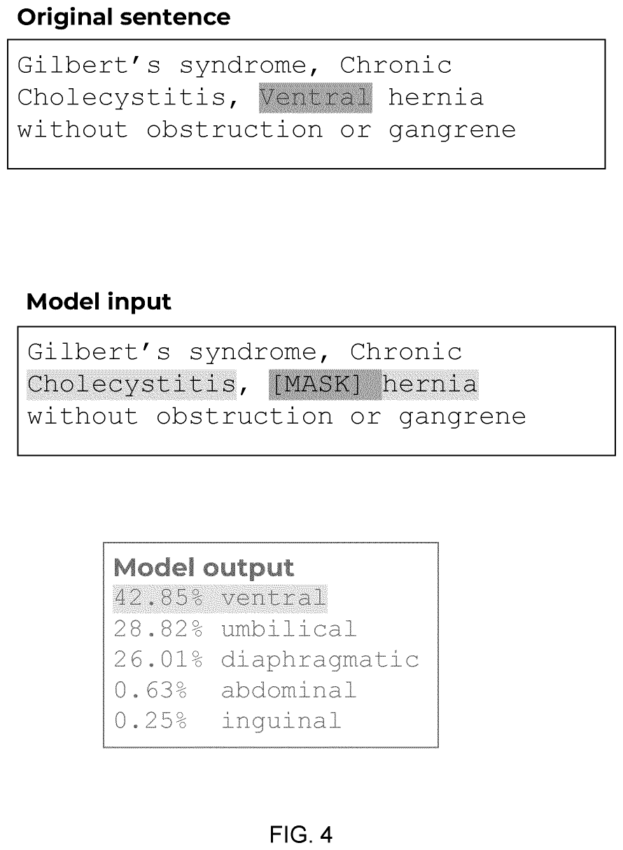

In particular, the training of the machine learning model involves two phases: a fine-tuning phase and a classification training phase. During the fine-tuning phase, the machine learning model (e.g. a BERT model that is pretrained on abstracts from PubMed and full-text articles from PubMedCentral) is trained on a masked language modelling task using the electronic health record data 200. Specifically, words from the text sequences 210 are masked at random, and each masked text sequence is provided to the machine learning model and the machine learning model is trained to predict the masked words. An example of the fine-tuning training is shown in FIG. 4, in which the term “Ventral” is masked in the text sequence, before the masked text sequence is input to the machine learning model for training.

During the classification training phase, the pre-trained and fine-tuned machine learning model is trained to predict a clinical outcome or characteristic based on the electronic health record data 200. For text sequences 210 positively labelled with a clinical outcome or characteristic, a first selection of words associated with the clinical outcome or characteristic are masked (also referred to as removed or deleted) from the text sequence 210. The masked text sequence is then provided to the machine learning model and the machine learning model is trained to predict a clinical outcome or characteristic based on the labelled masked text sequence.

Preferably, in addition to the masking of the first selection of words, a second selection of words associated with the clinical outcome or characteristic are replaced with a random word from a corpus of literature (e.g. biological literature or other words from text descriptions within the electronic health record data), whilst a third selection of words associated with the clinical outcome or characteristic are retained in the text sequence 210. For example, the first selection of words, the second selection of words, and the third selection of words may be selected with 80%, 10% and 10% respective probabilities. This modified text sequence is then provided to the machine learning model and the machine learning model is trained to predict a clinical outcome or characteristic based on the labelled modified text sequence. In this way, noise may be introduced into the training data which will ultimately increase the robustness of the model.

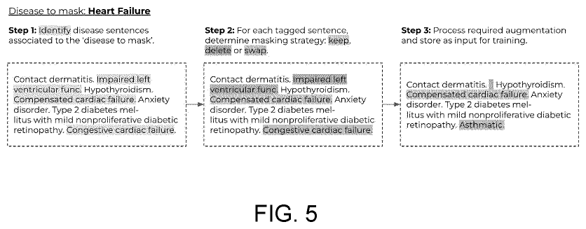

An example of the classification training is shown in FIG. 5, in which words associated with the disease diagnosis (i.e. the clinical outcome or characteristic) of “heart failure” are either deleted, swapped or kept within the text sequence 210, before the modified text sequence 210 is input to the machine learning model for training.

In the case of the text sequence 210 being positively labelled with two or more clinical outcomes or characteristics (i.e. the patient having comorbidities), a data augmentation strategy is employed. In particular, the text sequence 210 is copied to produce a number of duplicate text sequences 210 corresponding to the number of labelled clinical outcomes or characteristics, each duplicate text sequence being respectively associated with one of the two or more labelled clinical or characteristics. The duplicate text sequences 210 are then input into the machine learning model, and the training process described above (or modelling process described below) is performed on each of the duplicate text sequences 210. Each duplicate text sequence 210 is masked based on a respective clinical outcome or characteristic of the two or more labelled clinical outcomes or characteristics.

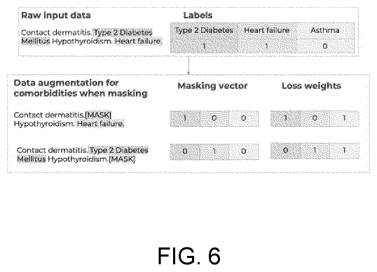

For example, as shown in FIG. 6, the text sequence 210 has been assigned labels corresponding to two clinical outcomes or characteristics: a first clinical outcome or characteristic (i.e. “type 2 diabetes”) and a second clinical outcome or characteristic (i.e. “heart failure”). The text sequence 210 is therefore duplicated to provide duplicate first and second text sequences 210. Words associated with the first clinical outcome or characteristic (i.e. “type 2 diabetes”) are masked in the first text sequence 210 and the masked first text sequence 210 is input to the machine learning model for training. Words associated with the second clinical outcome or characteristic (i.e. “heart failure”) are masked in second text sequence 210 and the masked second text sequence 210 is input to the machine learning model for training.

The masking of each of the duplicate text sequences 210 may be represented by masking vectors. A “1” value indicates that words associated with the corresponding clinical outcome or characteristic are to be masked, whereas a “0” value indicates that words associated with the corresponding clinical outcome or characteristic are not to be masked. Each masking vector can be used to define a loss weights vector, which is used in a loss function. As will be understood by the skilled person, the machine learning model is trained against the loss function (e.g. a masking binary cross entropy loss function). Words in the text sequence 210 which are associated with positively labelled clinical outcome or characteristic but are not being masked in the respective text sequence 210 will be assigned a loss weight of 0. Therefore, such words will not contribute to the loss function. For example, for the first text sequence 210 in FIG. 6 which is masked based on the clinical outcome or characteristic of “Type 2 diabetes”, the other labelled clinical outcome or characteristic of “Heart failure” (which is not masked) is assigned a loss weight of 0. In this way, it is possible to avoid overfitting the model to prevalent clinical outcomes or characteristics.

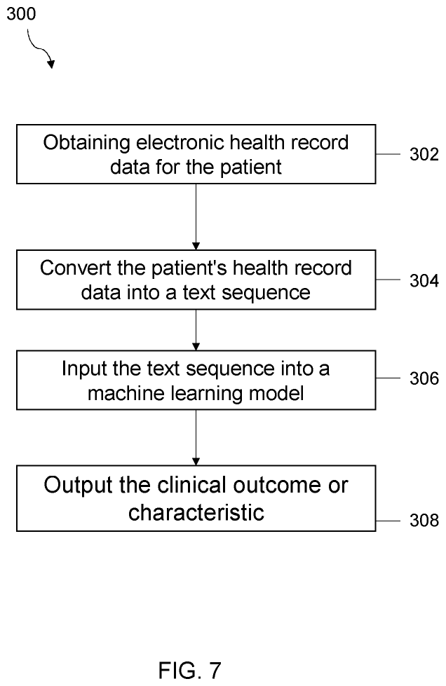

FIG. 7 illustrates a method 300 of predicting a clinical outcome or characteristic based on a patient's clinical history.

The method begins at step 302 by obtaining structured electronic health record data 200 for the patient. As previously discussed, the structured electronic health record data 200 comprises a plurality of clinical observations 202, each clinical observation 202 having a text description 204 and an associated time stamp 208.

At step 304, the patient's electronic health record data 202 is converted into a text sequence 210 by concatenating the text descriptions 204 in sequence of the time stamps 208. At step 306, the text sequence 210 is input into a machine learning model trained to predict a clinical outcome or characteristic based on the input text sequence 210, i.e. the text sequence 210 is input to a machine learning model trained based on method 100.

At step 308, the machine learning model outputs a predicted clinical outcome or characteristic based on the text sequence 210, and optionally also based on one or more continuous features associated with the text sequence 210. In particular, the machine learning model may be configured to provide a probability of the patient having the clinical outcome or characteristic.

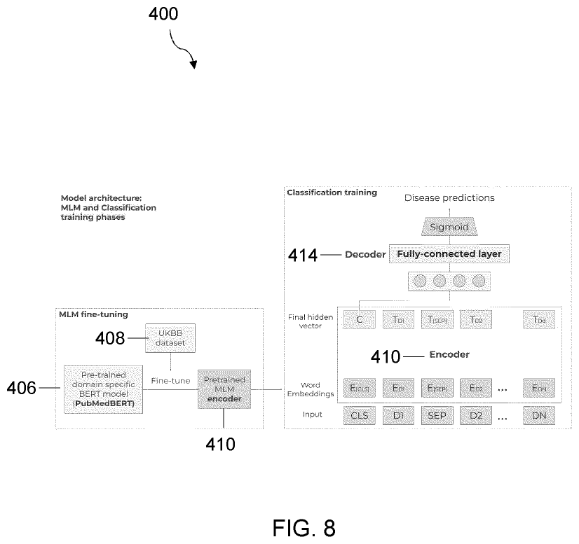

FIG. 8 is a schematic diagram illustrating an exemplary machine learning model architecture 400 according to the present invention. The model architecture 400 illustrates the two phases of fine-tuning and classification training described above.

The model architecture 400 includes a pre-trained machine learning model 404 (e.g. a BERT model that is pretrained using a masking objective on abstracts from PubMed and full-text articles from PubMedCentral) which is fine-tuned on a masked language modelling task (MLM) on the text sequences based on clinical observation descriptions. In this example, the text sequences 210 which are input to the pre-trained machine learning model 404 for fine-tuning are derived from the UK Biobank (UKBB) dataset, which is a large-scale biomedical database of around 500 k individuals between the ages of 40 and 54 at time of recruitment. The dataset includes rich genotyping and phenotyping data, both taken at recruitment and during primary and secondary care visits (GP and hospital). However, the skilled person will appreciate that the source of electronic health record data may vary. Following fine-tuning, the pre-trained (and fine-tuned) encoder 410 is used to train on the classification task.

The text sequences 210 are typically prepared for input to the encoder 410 by performing tokenisation on each text sequence 210 to generate a sequence of word piece tokens representing the sentence or multiple sentences of detect sequence. Any suitable tokenisation may be performed but in the present example BERT word-piece tokenisation is used to convert the text sequence 210 to word-piece tokens. As in the BERT architecture, each token sequence starts with the special token [CLS] denoting the start of a text sequence. [SEP] is used as a separator between different sentences when multiple input sentences are passed, whilst the masked words in the text sequence 210 are replaced with mask tokens [MASK] in the token sequence.

The sequence of tokens are then embedded (also referred to as encoded) into word embeddings (also referred to as text embeddings). Optionally, the word embeddings may be combined or concatenated with positional embeddings. The positional embeddings encode the position of the corresponding word piece token in the input text sequence 202. The word embeddings may also be concatenated with the one or more of the continuous features described previously. More particularly, the one or more continuous features may be mapped to one or more feature embeddings, which are then concatenated with the word embedding. For example, the word embeddings may be combined with age embeddings representing the patient's age at the time of the clinical observation 202.

In general, the word embeddings may be summed with the positional embeddings and/or the continuous feature embeddings to form an input representation. The encoder 401 is then used to map the input representation to a transformed output representation, i.e. a final hidden vector. The output representation is subsequently fed to a decoder 414, e.g. a fully connected linear layer, which then feeds into a sigmoid function to output probabilities for each clinical outcome or characteristic.

Further details relating to the above described methods are described below in accordance with one or more embodiments of the present invention.

### Language Modelling of Ehr Data for Ontology Agnostic Probabilistic Cohort Expansion

### Fusing Ontologies Via Text

The ontologies of GP and hospital records are made up of diagnostic codes (e.g. Read2/Read3 and ICD9/ICD10 codes, respectively) and their description. For each electronic health record (EHR) data source and associated ontology a ∈A, the set of concepts (e.g. diagnostic codes in the case of GP or hospital records) within this ontology may be denoted as Θₐ. The total vocabulary of concepts across all ontologies is denoted by Θ=U_{a∈A}Θa. For each patient, their full clinical history through time and across sources may be defined as the sequence of time-indexed concepts (θ₁, . . . , θₜ), θᵢ∈Θ, i=1, . . . , t.

The approach of the present invention relies on the assumption that for every concept θ∈Θ, there exists a unique text description ξ_{θ}∈Ξ. For example, under the ICD10 ontology the alphanumeric code E11.9 has the associated description ‘Type 2 diabetes mellitus without complications’. Thus for each patient, their clinical history represented as a sequence of concepts can be uniquely represented by the concatenation of sequences of clinical descriptions (ξ₁, . . . , ξₜ), ξᵢ∈Ξ, i=1, . . . , t, ordered in time. As discussed previously, FIG. 2 shows an example of such a text sequence 210 fused across GP and hospital record ontologies.

### Model Design

To form the input to the machine learning model, the raw text sequence 210 of code descriptions are processed into tokens (e.g. words and subwords). For example, the tokens X=W(ξ₁, . . . , ξₜ)=(x₁, . . . , xₙ) may be formed under a fixed size vocabulary V with the tokenizer W (e.g. using a Word-Piece tokenizer).

Let Δ={d₁, . . . , d_{D}} denote an ordered set of unique clinical outcomes (or characteristics) dᵢ. It is assumed that for each outcome dᵢ∈Δ, there exists an indicator function 1_{di} that assigns a binary label to individuals according to the presence or absence of dᵢ. An example is described in the next section.

Let X^{(p)}=(x₁^{(p)}, . . . , xₙ^{(p)}) denote the tokenized input sequence of individual p. It forms the input an encoding function x₁^{(p)}, . . . , xₙ^{(p)}=Encoder(X^{(p)}), where each xᵢ is a fixed-length vector representation of each input token xᵢ.

Let y^{(p)}=(y₁^{(p)}, . . . , y_{D}^{(p)}), yᵢ^{(p)}∈{0, 1}, be the individual's phenotype labels representing presence or absence of outcomes or characteristics d₁, . . . , d_{D}. Given a learned representation over inputs, y^{(p)} is decoded under the predictive model P(y^{(p)|}X^{(p)}). The probability of each outcome or characteristic dᵢ is calculated given the input sequence encoding P(yᵢ^{(p)}|x₁^{(p)}, . . . , xₙ^{(p)}) via a decoder module. Specifically, the representation is decoded into logits per outcome z₁^{(p)}, . . . , z_{D}^{(p)}=Decoder(x₁^{(p)}, . . . , xₙ^{(p)}) and calculate the probability per outcome as P(y_{d}^{(p)}|x₁^{(p)}, . . . , xₙ^{(p)})=σ(z_{d}^{(p)}), where σ denotes the sigmoid function.

For ease of reading, the superscript (p) denoting the sample index will be omitted in the remainder of the description.

### Label Generation: Oracle Feature Tagging for Disease Phenotyping

Given a set of diagnostic codes Θ and text descriptions Ξ, external oracles are used to assign labels for a given set of target outcomes or characteristics Δ. It is assumed that for each clinical outcome or characteristic d∈Δ there exists a mapping 1_{d}: Θ×Ξ→{0, 1}, (θ, ξ)→δ_{d} indicating whether the presence of d can be inferred from the code and its description. An aggregated clinical outcome or characteristic label of 1 is assigned, if 1_{d}(θ, ξ)=1 for any of the code-description pairs (θ, ξ) in the input sequence, and 0 otherwise. An example of how a unified clinical history of an individual is mapped to a multi-hot label y is shown in FIG. 3.

In one example, Δ is a set of disease phenotypes, for example the CALIBER phenotype definitions which are collections of hand-crafted diagnostic codes across primary and secondary care ontologies for general phenotypes, or disease-specific phenotyping algorithms.

Data Augmentation with Clinical Masking

Input descriptions ξ from code-description pairs (θ, ξ) with 1d(θ, ξ)=1 for d∈Δ are masked using the following masking strategy during training and validation. During testing, these code-descriptions pairs are fully removed from the input sequence.
1. Full Mask (with 80% probability): remove ξ.
2. Random Replacement (with 10% probability): replace ξ with a randomly selected description from the corpus.
3. 3. Keep (with 10% probability): retain ξ.

A worked example is shown in FIG. 5.

### Data Augmentation for Comorbidities

The masking approach described above is straightforward if an individual has only one positive label, but many people have comorbidities, e.g. co-occurring conditions that are often well-known risk factors or complications. To allow for comorbidities in the input sequence, a data augmentation strategy is employed. For a sample with multiple positive labels dᵢ₁, . . . , dᵢₙ, n input samples are created by duplicating both the input sequence and target vector of phenotype labels. The jth duplicated input sequence is masked with the masking strategy for clinical outcome or characteristic dᵢⱼ described previously, e.g. ξ is masked if 1_{d}(θ, ξ)=1, where d=dᵢⱼ. It is described in the next section how the contribution of the target vector y is augmented to the loss function. To do this, a binary masking vector y^{ij}=(γ₁^{ij}, . . . , γ_{D}^{ij}) where γⱼ^{ij}=1 and γₖ^{ij}=0, k=1, . . . , D, k≠j. Individuals with no positive clinical outcome or characteristic labels are assigned an all-zero masking vector.

### Defining a Loss Function in the Presence of Comorbidities and Varying Prevalences

Since the model is configured to predict over all clinical outcomes or characteristics simultaneously, there is a risk of overfitting the model when an input sequence contains descriptions that are strongly associated to positive clinical outcome or characteristic labels but that are not identified with the indicator functions 1_{d} for d∈Δ and subsequently masked. For a given input text sequence X, target label vector y=(y₁, . . . , y_{D}) and masking vector γ=(γ1, . . . , γD), the following loss weights may advantageously be used:

$${ω}_{d} = \{ \begin{matrix}1 & if {y}_{d} = 0 \\ 1 & if {y}_{d} = 1 and {γ}_{d} = 1 \\ 0 & if {y}_{d} = 1 and {γ}_{d} = 0\end{matrix} . \qquad ( 1 )$$

Disease prevalences can vary significantly, making prediction classes highly imbalanced in the practical setting. For cohort expansion, it is desirable to increase recall while balancing a decline in precision. The positive weight ρ_{d} is defined as:

$${ρ}_{d} = \frac{{TN}_{d}}{{TP}_{d}} , for all d ∈ Δ . \qquad ( 2 )$$

The loss function can then be defined as a mean-reduced binary cross-entropy loss function over clinical outcomes or characteristics, where differing clinical outcome or characteristic prevalence and present comorbidities are handled with positive example weights and loss weights to avoid overfitting to prevalent clinical outcomes or characteristics, or those with many highly associated diagnostic codes and descriptions:
where σ denotes the sigmoid function, ω_{d} the comorbidity-derived loss weight (Equation 1), β_{d} the positive weight (Equation 2), and z_{d}^{(p)} the predicted probability for clinical outcome or characteristic d∈Δ for sample (e.g. individual) p.

$${ℒ}^{( p )} = - \frac{1}{\vert Δ \vert} {\sum }_{d ∈ Δ} {ω}_{d}^{( p )} ( {ρ}_{d} {y}_{d}^{( p )} log σ ( {𝓏}_{d}^{( p )} ) \qquad ( 3 )$$

$$+ ( 1 - {y}_{d}^{( p )} ) log σ ( 1 - {𝓏}_{d}^{( p )} ) ) ,$$

### Example of Specific Implementation of Model and Results

In the below section, a specific implementation of the model and the results of its performance is disclosed.

As a proof-of-concept we chose four diseases that differ in terms of prevalence and clinical characteristics: type 2 diabetes mellitus (T2DM) is one of the most prevalent chronic diseases in the UK, and is mainly followed in the primary care setting exemplifying the need for the usage of heterogeneous data sources ( ); heart failure (HF) is one of the main causes of death in the older population and has several risk factors and associated comorbidities ( ); malignant neoplasms of the breast and of the prostate are both less prevalent diseases almost exclusively present in only biologically females or males, respectively (Ly et al., 2013; Rawla, 2019). We test the performance of our model on its ability to diagnose cases, compare it to other methods, and clinically validate the predictions on T2DM with available orthogonal data.

### Data

The UK Biobank (UKBB) (Sudlow et al., 2015) is a large-scale biomedical database of around 500 k individuals between the ages of 40 and 54 at time of recruitment. It includes rich genotyping and phenotyping data, both taken at recruitment and during primary and secondary care visits (GP and hospital). We use patient records from GP and hospital visits in the form of code ontologies Read2, Read3, ICD9, and ICD10 together with their textual descriptors. To avoid bias towards more acute events that are usually present in hospital, we restrict the data set to individuals that have both hospital and GP records, which reduces our cohort to 154, 668 individuals. We use phenotype definitions from CALIBER (Kuan et al., 2019) to label patients with T2DM, HF, malignant neoplasm of the breast, and malignant neoplasm of the prostate.

### Model Training

We use the pretrained language model Pub-MedBERT (Gu et al., 2020) as encoder of the tokenised input sequences of code descriptions. Since our input systematically differs from the general scientific text on which Pub-MedBERT was trained, we fine-tuned on the masked-language modelling (MLM) task, by masking words (e.g. code descriptions) at random following the original BERT paper (Devlin et al., 2018). The model, fine-tuned using the full UKBB cohort of 138,079 patients, was trained with early stopping for epochs with a batch size of 32 and a learning rate of 4×10⁻⁵ using gradient descent with an AdamW optimizer, and weight decay of 0.01. The output dimension of the encoder was 768.

The proposed LMPCE model is using the fine-tuned encoder and a fully connected linear layer as decoder. The model architecture is described in more detail in FIG. 8 previously described. To train on the multi-label classification task of outcome prediction, we split the data into training, validation and test sets with a 60/20/20 split and follow the clinical masking strategy (e.g. as described in the “Data Augmentation with Clinical Masking” section). We use 5-fold cross-validation on the training set to train a total of five models for 3 epochs on five equally sampled folds f₀, . . . f₄, holding back folds fᵢ for validation and f_{(i+1) mod 5} for testing for model i, i=1, . . . , 5. We use the stratified sampling method to maintain the same phenotype proportion in every split (Sechidis et al., 2011). We used a learning rate of 10⁻⁵, and a warm-up proportion of 0.25. Performance on the full validation set was monitored every 0.25 epochs.

### Model Evaluation

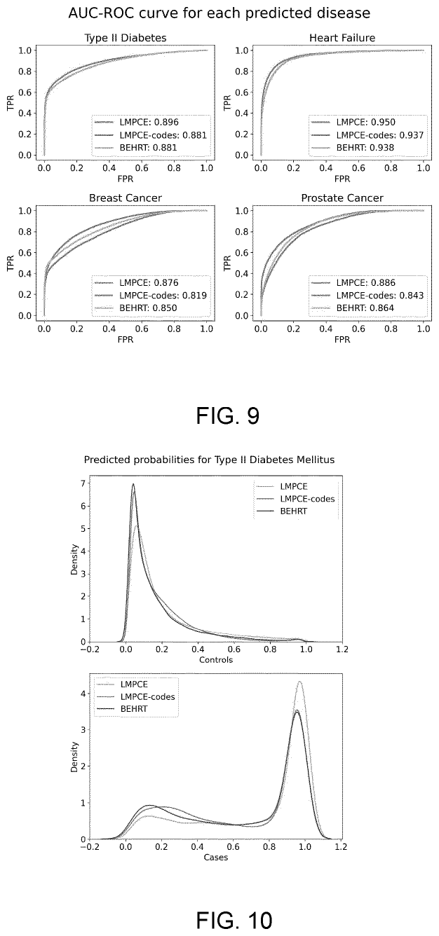

We compare performance of our model LMPCE to BEHRT (Li et al., 2019). BEHRT takes a tokenised sequence of diagnostic codes, age and position embeddings as input. Code ontologies from hospital and GP records are mapped to CALIBER definitions (Kuan et al., 2019), removing unmapped codes. A transformer model is pre-trained to predict masked diagnostic code tokens before it is trained to predict a set of possible diagnoses an individual may develop given the input sequence. We trained such a BEHRT model to predict an individual developing the four phenotypes with a small change to the token set: phenotype definitions in CALIBER include different categories (for example, phenotype ‘diabetes’ contains categories ‘type 1’ and ‘type 2’) that were ignored by the original BEHRT publication, but we define a token per CALIBER phenotype and category. We also trained an LMPCE model restricted to CALIBER code tokens (denoted LMPCE-codes) for comparison. LMPCE shows the best performance across all four phenotypes in terms of recall at 0.5 and AUC on the test set, as shown in Table 1 and FIG. 9. BEHRT performs slightly better than LMPCE-codes, indicating the benefit of adding visit position and age. Performance varies across phenotypes, presumably due to different clinical characteristics making some diseases easier to predict than others.

TABLE 1
Average and phenotype specific recall at 0.5
on the UKBB test data set for each method.

| | | | Breast | Prostate | Average |
|---|---|---|---|---|---|
| | T2D | HF | Cancer | Cancer | Recall |
| LMPCE-codes | 0.641 | 0.755 | 0.415 | 0.356 | 0.542 |
| BEHRT | 0.658 | 0.773 | 0.478 | 0.408 | 0.579 |
| LMPCE | 0.747 | 0.851 | 0.555 | 0.578 | 0.683 |

Patients without a diagnosis in the data set (referred as controls) that are predicted as having high probability of disease may represent missed cases. After inspection of the distributions of the predicted probabilities, we defined sets of missed cases as controls with a predicted probability in the 98th percentile for each of the methods and phenotypes. To assess LMPCE as a cohort expansion method, we will evaluate the characteristics of these groups in context of the different phenotypes in more depth in the next sections.

### Evaluation on Sex-Specific Diseases

We included two cancer types that are specific or more common in populations with the same biological sex. Such a label is not present in the input data. To evaluate whether LMPCE would be able to infer such underlying characteristics, we compared the percentages of females and males in the set of predicted missed cases with the percentages of female and male diagnosed cases in the cohort across the methods for each cancer type. While LMPCE-codes captured better the female and male proportions in heart failure and T2DM, only LMPCE was able to better recover the sex-specificity of breast and prostate cancer (Table 2) with both BEHRT and LMPCE-codes predicting more missed male than female cases of breast cancer.

TABLE 2
Prevalence of female and male patients per group. Cases display
patients with known diagnosis in the cohort. LMPCE-codes,
BEHRT and LMPCE (ours) show controls with a predicted probability
above the 98th percentile for each phenotype.

| | T2DM | | HF | | Breast Cancer | | Prostate Cancer | |
|---|---|---|---|---|---|---|---|---|
| | female | male | female | male | female | male | female | male |
| Cases | 40.32 | 59.68 | 33.82 | 66.18 | 99.2 | 0.8 | 0 | 100 |
| LMPCE-codes | 40.69 | 59.31 | 35.46 | 64.54 | 42.66 | 57.34 | 48.32 | 51.68 |
| BEHRT | 41.44 | 58.56 | 36.55 | 63.45 | 43.56 | 56.44 | 41.25 | 58.75 |
| LMPCE | 41.61 | 58.39 | 37.93 | 62.07 | 70.13 | 29.87 | 25.38 | 74.62 |

### Clinical Evaluation on Type 2 Diabetes Mellitus

T2DM lends itself as a use case to qualitatively evaluate the predictions of missed cases as it is a well studied, slowly developing disease with varying disease severity. Disease-specific external and orthogonal data are readily available.

The predicted probabilities for all individuals in the data set follow an expected bimodal distribution separating cases and controls (FIG. 10). We used thresholds based on percentiles of LMPCE's predicted probabilities of T2DM to define five different groups shown in Table 3 for further investigation.

Predicted Probabilities Correlate with a Measures of Disease Severity

TABLE 3
Case and control cohorts and groups of interest
based on LMPCE's predicted probability for T2DM.

| Patient group | Size |
|---|---|
| Cases | 16431 |
| Controls | 113501 |
| Controls with high probability | 2020 |
| (p >= 0.85, 98th percentile) | |
| Cases with high probability | 2072 |
| (p >= 0.985, 90th percentile) | |
| Cases with low probability | 2343 |
| (p <= 0.25, 12th percentile) | |

Haemoglobin A1c (HbA1c) is a blood biomarker used to diagnose and to define the severity of diabetes in the clinic with the following UK guidelines: healthy below 42, prediabetes between 42 and 47 and diabetes 48 mmol/mol or over. The input data did not include biomarkers, so we can use it for evaluation.

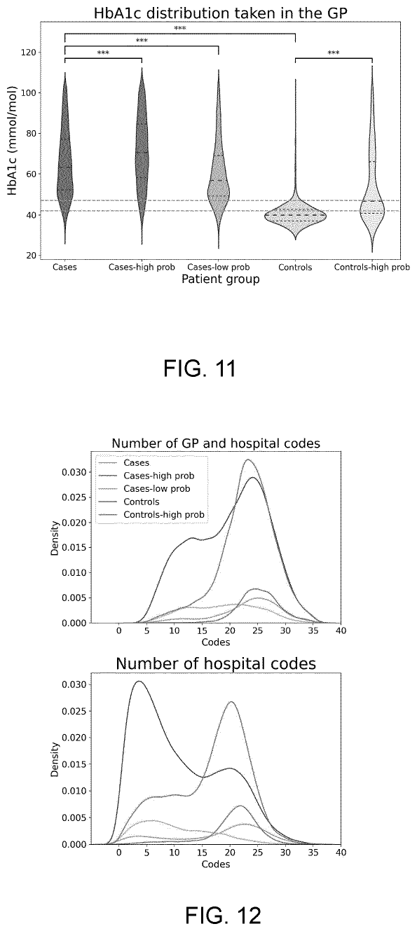

To define a single value per patient, we use the 95-th percentile of their HbA1c measurements in the GP data. Cases that LMPCE identified with high probability had the highest HbA1c mean levels when compared to the previously identified groups (FIG. 11). Cases identified with low probability were in the prediabetic range of HbA1c levels, possibly indicating that their diabetes is controlled through treatment. Missed cases (e.g. controls predicted to have T2DM with high probability) had elevated HbA1c levels close d the prediabetic stage when compared to all controls, representing individuals at risk of developing T2DM.

We investigated the association of the predicted probabilities of having T2DM with several other measures of disease severity: the number of GP and hospital visits, survival, and cardiovascular risk.

### Number of GP Visits and Hospital Admissions

As expected, both cases and controls with a high predicted probability of a T2DM diagnosis, exhibit a slightly higher number of GP and hospital visits than the other groups (FIG. 12), indicating that they are experiencing a more severe form of T2DM requiring care. This is particularly dramatic in the case of hospital visits, indicating patients experiencing acute events: both cases and controls with a high predicted probability visit a hospital approximately 10 more times than their low probability counterparts. Although the model was not given information from which data source the input data was coming from, this analysis indicates that it has learned to associate acute events with disease severity T.

### Survival Analysis

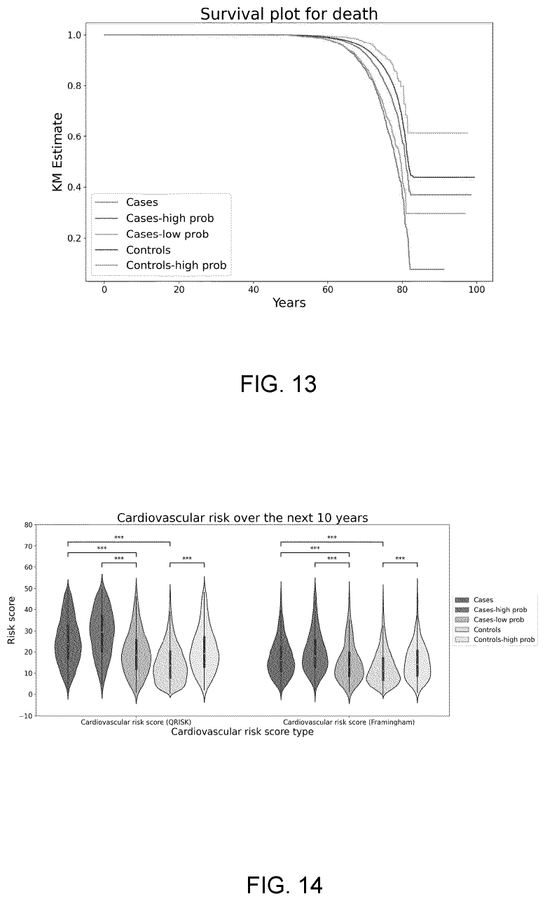

To compare the survival across different groups of individuals, we use the Kaplan-Meier estimator with all-cause death as the endpoint with right-censored data (e.g. if a patient is alive without any event occurrence since the last follow-up). Both cases and controls with high predicted probability had the lowest survival, followed by general cases, controls and finally cases with low predicted probability (FIG. 13), indicating that the model's predicted probability is associated with disease severity and ultimately survival.

### Cardiovascular Risk

T2DM is a known risk factor and comorbidity of cardiovascular disease, which, in turn, is the most prevalent cause of death in T2DM patients. The GP records contain Framingham and QRISK scores, these are two scores that assess an individual's risk of developing cardiovascular disease within the next 10 years, based on several coronary risk factors. The Framingham score is derived from an individual's age, gender, total cholesterol, high density lipoprotein cholesterol, smoking habits, and systolic blood pressure, whereas the QRISK score extends this score with additional factors such as body mass index, ethnicity, measures of deprivation, chronic kidney disease, rheumatoid arthritis, atrial fibrillation, diabetes mellitus, and antihypertensive treatment. Both cases and controls with high predicted probability of having T2DM had a higher risk of developing cardiovascular disease compared to their low predicted probability counterparts (FIG. 14) indicating that the model has learned to associate the risk of developing both diseases at the same time.

Taken together, our results show that LMPCE's predicted probabilities of being diagnosed with T2DM is associated with disease severity across different measures.

Polygenic Risk Scores Align with Predicted Probabilities Across Cases and Controls

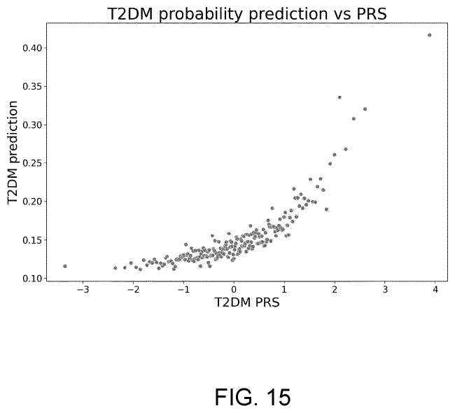

Genetic risk for complex diseases like T2DM arise from many genetic changes that, when taken together, can increase an individual's risk of developing the disease. To measure this combinatorial risk or genetic predisposition, polygenic risk scores (PRS) have been developed for a suite of diseases. Sinnott-Armstrong et al. (2019) developed PRS for 35 blood and urine biomarkers based on the UK Biobank participants and combined those into multi-PRS for a set of diseases, including T2DM. We computed and standardised the PRS for T2DM across all individuals in our cohort (Lewis and Vassos, 2017). A higher predicted probability of T2DM was associated with a higher genetic risk (FIG. 15).

### CONCLUSION

We have developed an ontology-agnostic method for probabilistic cohort expansion. Our approach fuses primary and secondary care data via text, and we propose a data augmentation approach to deal with the presence of comorbidities in a patient's history. Our evaluations suggest that our method identifies currently undiagnosed patients better than non-text and single ontology approaches and that the predicted probability is associated with disease severity.

The following details further application(s) for the trained machine model(s) described above with reference to FIGS. 1 to 15.

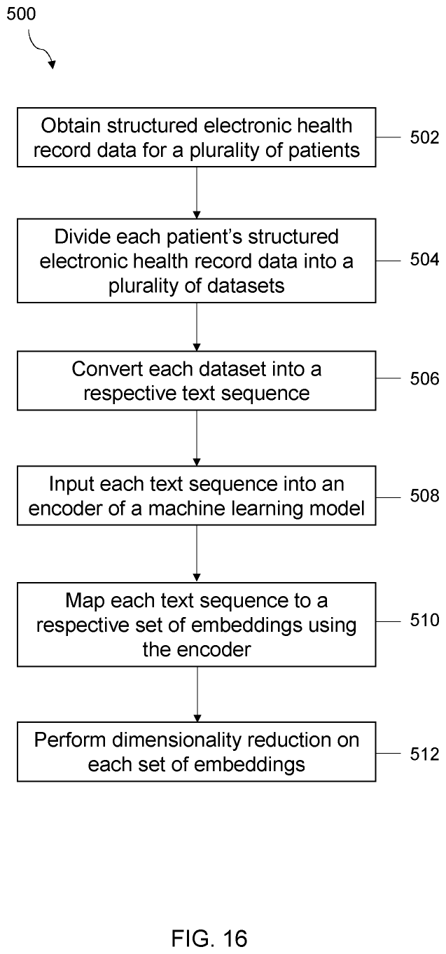

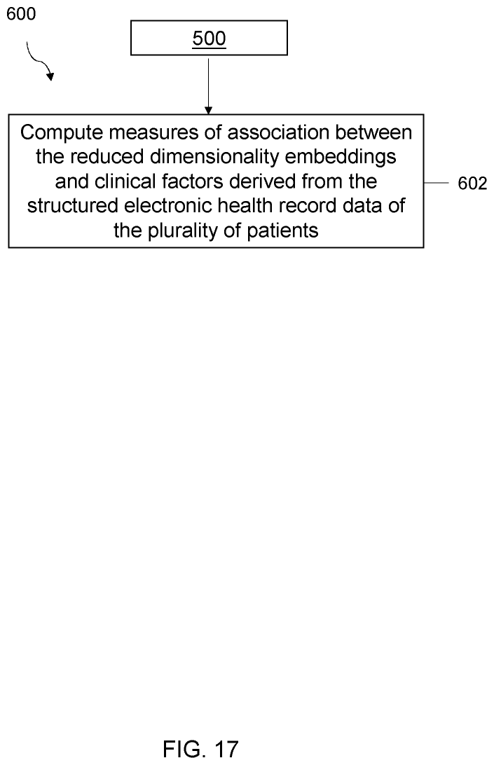

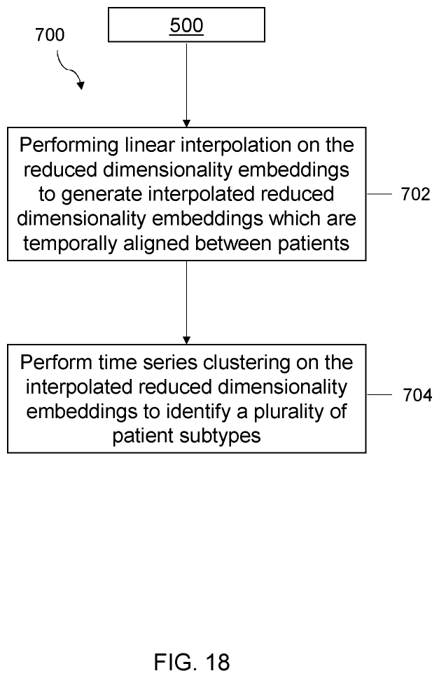

FIG. 16 illustrates a method 500 for generating dimensionality reduced embeddings which capture and represent disease stages or themes within a patient's medical history. FIGS. 17 and 18 illustrate methods 600 and 700 respectively which utilise the dimensionality reduced embeddings produced by method 500 in order to provide clinically meaningful insight into disease stages and patient subtypes, as will be discussed in further detail below. It will be appreciated that method 500 may be combined with one or both of methods 600 and 700.

Method 500 begins at step 502 wherein structured electronic health record data is obtained for a plurality of patients. As described previously, the structured electronic health record data for each patient includes a plurality of clinical observations, with each clinical observation having a text description and an associated time stamp. Preferably, the plurality of patients associated with the structured electronic health record data have all been diagnosed with the same particular disease.

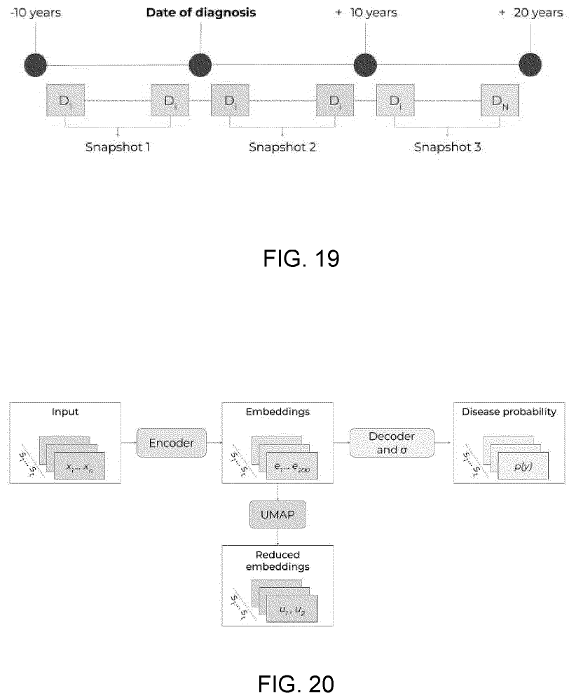

At step 504, each patient's electronic health record data is split into a plurality of datasets. The datasets may also be referred to as snapshots Si, and each include a plurality of clinical observations spanning a particular time period. Each dataset therefore represents the clinical history of a patient over a particular time period. The time period can be defined with respect to the patient's date of diagnosis for the particular disease. This is illustrated in FIG. 19 which provides a schematic diagram of EHR data which has been divided into three snapshots. Snapshot 1 includes clinical observations from 10 years prior to the date of the diagnosis up to the date of the diagnosis, snapshot 2 includes clinical observations from the date of diagnosis to 10 years after the date of diagnosis, and snapshot 3 includes clinical observations from 10 years after the date of diagnosis to 20 years after the date of diagnosis.

Advantageously, by dividing the EHR data of each patient into a plurality of dataset, this allows for the downstream characterization of disease progression and temporal changes within the patient's medical data. The datasets may be restricted such that the datasets do not include any clinical observations which are associated with the particular disease. The datasets may also be restricted such that the datasets only include clinical observations which are diagnoses of diseases.

At step 506, each dataset is converted into a text sequence which is a concatenation of the text descriptions ordered in time, i.e. in sequence of the time stamps.

At step 508, each text sequence is input into an encoder of a machine learning model. The text sequences may be input into the encoder of the trained machine model(s) described previously with reference to FIGS. 1 to 15, which has been trained to predict a clinical outcome or characteristic based on a patient's clinical history. More specifically, the encoder of the machine learning model has been trained to learn representations (e.g. embeddings) that encode the semantics of the text sequences comprising clinical observation descriptions. This is referred to above as the fine tuning phase of training. The machine learning model (and in particular the encoder) may be trained based on the datasets described above, e.g. datasets which do not include any clinical observations which are associated with the particular disease and only include clinical observations which are diagnoses of diseases.

Of course, the skilled person will appreciate that the method 500 is not limited for use with the specific trained machine learning model(s) and encoder(s) described above, and the method 500 may operate based on other machine learning models and encoders which have been trained to generate embeddings that encode the semantics of text sequences associated with clinical observations, and preferably in which the machine learning model has been trained to predict a clinical outcome or characteristic based on the embeddings.

As described previously, the text sequences are typically prepared for input to the encoder by performing tokenisation on each text sequence to generate tokens (word and sub-word pieces) that may be transformed into embeddings by the encoder. For example, each tokenized input sequence X may be defined by X=(x₁, . . . , xₙ) wherein n is the tokenized sequence length. That is, the text sequences may be input into the encoder in the form of tokenized input sequences.

At step 510, each text sequence input into the encoder is transformed into a set of embeddings by the encoder. For example, the tokenized input sequence may form the input to an encoding function e₁, . . . , eₙ=Encoder (X) wherein eᵢ is a fixed length vector representation of each input token xᵢ. Accordingly, as the machine learning model has been trained to identify disease-specific representations from each text sequence to classify disease, the resulting embedding space will be understood to represent different disease stages or themes.

At step 512, each set of embeddings is dimensionality reduced to produce reduced dimensionality embeddings. By reducing the dimensionality of the embeddings, the clinical interpretability of the embedding space is improved thereby allowing clinicians to obtain clinically meaningful insight in regard to the particular disease.

In one example, each set of embeddings may be reduced to a two-dimensional vector. That is, each set of embeddings e₁, . . . , eₙ can be reduced to a two-dimensional vector U=u₁, u₂, wherein u₁ is a first component and u₂ is a second component of the two-dimensional vector. However, the skilled person will appreciate that the dimensionality reduced embeddings are not limited to two-dimensional vectors, and in alternative examples the dimensionality reduced embeddings may include more than two components, e.g. three components.

In one example, the dimensionality reduced embeddings may be generated using the Uniform Manifold Approximation and Projection (UMAP) algorithm. In other examples, the dimensionality reduced embeddings may be generated using alternative dimensionality reduction algorithms.

FIG. 20 is an exemplary model flow diagram providing further illustration of the steps of method 500 with reference to an exemplary model architecture. The machine learning model includes an encoder and a decoder, and has been trained to calculate disease probability p(y) based on electronic health record data as previously described. The snapshots s₁, . . . , sₜ which are each represented by tokenized sequences x₁, . . . , xₙ are input into the model and the encoder of the model transforms each tokenized sequence into a set of embeddings, e.g. e₁, . . . , e₂₀₀. Each set of embeddings is then dimensionality reduced, e.g. using UMAP, to generate reducing embeddings, e.g. a two-dimensional vector u₁, u₂. For example, performance of the method 500 results in a first set of embeddings (e.g. e₁, . . . , e₂₀₀) corresponding to a first snapshot s₁ being reduced to a single two-dimensional vector (e.g. u₁, u₂).

FIG. 17 illustrates a method 600 which is a continuation of the method 500. Method 600 is aimed at evaluating the separation of disease stages in the embedding space by assessing the association between the reduced embeddings and certain clinical factors.

In particular at step 602 measures of association are computed between the reduced dimensionality embeddings and clinical factors extracted from the structured electronic health record data of the plurality of patients. That is, for each clinical factor, a measure of association is calculated between said clinical factor and the reduced embeddings. The term clinical factors will be understood to refer to any clinically-relevant marker identified in the structured electronic health record data, such as symptoms, laboratory results, vital signs, prescription medication, and other co occurring conditions (comorbidities).

In one example, the measures of association may comprise or consist of point-biserial correlation coefficients. The point-biserial correlation coefficient provides a measure of the strength of association between a continuous variable (e.g. a component of the reduced embeddings) and a binary variable (e.g. the clinical factor, which is either identified as being present or absent from the EHR data from which each snapshot is derived). For example, for each clinical factor fₖ, a first point-biserial correlation coefficient r_{f}_{ₖ}ᵤ_{₁} is calculated between the clinical factor fₖ and the first components u₁ of the two-dimensional vectors across all datasets, and a second point-biserial correlation coefficient r_{f}_{ₖ}ᵤ_{₂} is calculated between the clinical factor fₖ and the second components u₂ of the two-dimensional vectors across all datasets. In particular, r_{f}_{ₖ}ᵤ_{₁} can be defined as follows:

$${r}_{{f}_{k} {u}_{1}} = \frac{\underline{{Y}_{1}} - \underline{{Y}_{0}}}{{s}_{y}} \sqrt{\frac{{N}_{1} {N}_{0}}{N ( N - 1 )}}$$

wherein Y₁ is the mean of the first components u₁ which contain the clinical factor fₖ in their corresponding EHR data, Y₀ is the mean of the first components u₁ which do not contain the clinical factor fₖ in their corresponding EHR data, N₁ is the number of first components u₁ which contain the clinical factor fₖ in their corresponding EHR data, N₀ is the number of first components u₁ which contain the clinical factor fₖ in their corresponding EHR data, N is the total number of first components u₁ (i.e. corresponding to the number of snapshots), and s_{y} is the standard deviation of the first components u₁. r_{f}_{ₖ}ᵤ_{₂} can be similarly defined mutatis mutandis.

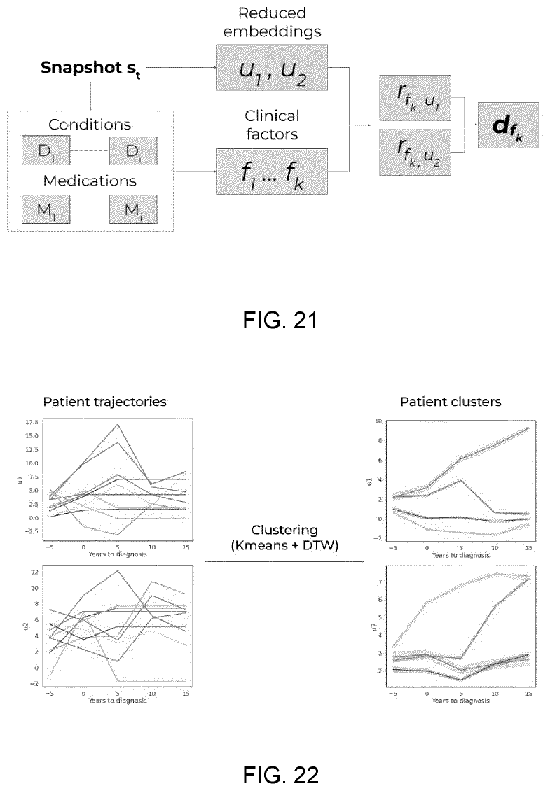

Optionally, the L2 norm (Euclidean distance to the origin) may also be calculated for each clinical factor fₖ, as d_{f}_{ₖ}=\sqrt{r²_{f}_{ₖ}ᵤ_{₁}+r²_{f}_{ₖ}ᵤ_{₂}}, wherein 0 indicates no correlation between fₖ and (u₁, u₂). The first point-biserial correlation coefficient, the second point-biserial coefficient, and the L2 norm may each be considered as a measure of association for a clinical factor. The measures of associations may be evaluated for different clinical factors to identify disease themes and disease stages. The calculation of measures of association for each snapshot sₜ is further illustrated in FIG. 21. It will be appreciated that additional point-biserial correlation coefficients (e.g. a third point-biserial correlation coefficient) may be calculated in the case of the reducing dimensionality embedding comprising more than two components.

FIG. 17 illustrates a method 700 which is a continuation of the method 500 and/or method 600. Method 600 is aimed at identifying patient subtypes based on a plurality of patients' clinical histories. As used herein, the term “patient subtypes” will be understood to refer to subpopulations of clinically related patients. The identification of patient subtypes may also be referred to as classifying patients into clinically relevant subgroups or clinical patient groups sharing common biological mechanisms.

At step 702, the reduced dimensionality embeddings are linearly interpolated to generate interpolated reduced embeddings which are temporally aligned between patients. That is, each dataset (and thus each set of reduced embeddings) is associated with a respective time period defined relative to the patient's date of diagnosis. However, the temporal positions of the time periods relative to the date of diagnosis may vary between different patients. Linear interpolation is therefore performed on reduced dimensionality embeddings to produce interpolated reduced embeddings which are associated with consistent time steps across all patient data.

For example, referring to FIG. 19, the temporal position of each of snapshots 1 to 3 may be defined by the midpoint of its time period (e.g. −5, 5, 15). Thus, if a consistent time step of 5 years is desired, the reduced dimensionality embeddings corresponding to snapshots 1 to 3 can be linearly interpolated to generate interpolated reduced dimensionality embeddings associated having a time step of 5 years (e.g. −5, 0, 5, 10, 15). In practice, this would mean that additional reduced dimensionality embeddings associated with 0 years and 10 years respectively will be generated. The reduced dimensionality embeddings corresponding to other patients can be similarly interpolated. It will be appreciated that, in the case that the reduced dimensionality embeddings across the plurality of patients are already associated with a consistent time step, interpolation may not be required.

At step 704, time series clustering is performed on the interpolated reduced dimensionality embeddings, thereby resulting in the identification of patient clusters corresponding to clinical subgroups. In this way, disease progression patterns may be identified, which facilitates improved medical decision making and treatment plans. The clusters may be clinically characterised and evaluated based on the method 600, e.g. by evaluating the association between clinical factors and the reduced embeddings in each cluster.

In one example, the interpolated reduced dimensionality embeddings may be clustered using a k-means algorithm, preferably with multivariate dynamic time warping (DTW). In another example, the interpolated reduced dimensionality embeddings may be clustered using a hierarchical clustering algorithm.

The skilled person will appreciate that the number of clusters may be pre-selected based on the use-case of the method. In one example, four clusters may be selected.

The following describes an example implementation of the trained machine learning model, and in particular the encoder, described above.

### Interpreting Deep Embeddings for Patient Progression Clustering

### Input Generation

Medical ontologies are the basic building block of how structured EHR data are recorded but each healthcare setting (e.g. primary care or secondary care) uses a different ontology (NHS). Medical ontologies are hierarchical data structures which contain healthcare concepts that enable healthcare professionals to record information consistently. Ontology concepts consist of a unique identifier and the corresponding description (for example, J45-Asthma is a code-description pair in the ICD10 ontology used in hospitalisation EHR). For each patient, we defined their entire clinical history as the concatenation of sequences of clinical descriptions (ξ_{θ1}, . . . , ξ_{θt}), ξ_{θi} ∈Ξ_{θ}, i=1, . . . , t, ordered in time (Munoz-Farre et al., 2022) across multiple EHR sources, with Ξ_{θ} being the set of descriptions for each ontology θ. To capture temporal patterns and changes in disease progression, we slice each patient's history into “snapshots” around the date of diagnosis (e.g. see FIG. 19 which illustrates an example of constructing 10 year snapshots from EHR data). For each snapshot, we process the raw text sequence of descriptions into tokens (word and sub-word pieces), using a tokenizer W as X=W (ξ_{θ1}, . . . , ξ_{θt})=(x₁, . . . , xₙ), with n as the tokenized sequence length.

### Model Design

We trained a model that classifies disease based on EHR sequences. Let X^{(p,s)}=(x₁^{(p,s)}, . . . , xₙ^{(p,s)}) denote the tokenized input sequence of an individual p and a snapshot s. It forms the input to an encoding function e₁^{(p,s)}, . . . , eₙ^{(p,s)}=Encoder(X^{(p,s)}), where each eᵢ is a fixed-length vector representation of each input token xᵢ. Let y^{(p)} ∈{0, 1} be the disease label. To calculate disease probability P(y^{(p,s)}|X^{(p,s)}), the embeddings of the CLS token are fed into a decoder z₁^{(p,s)}, . . . , z_{D}^{(p,s)}=Decoder(e₁^{(p,s)}, . . . , eₙ^{(p,s)}), and the resulting logits are fed into a softmax function σ P (y^{(p,s)}|e₁^{(p,s)}, . . . , eₙ^{(p,s)})=σ(z^{(p,s)}) (e.g. see FIG. 20 illustrating a model diagram flow. Snapshot sequences are tokenized to generate the input, which is fed into the encoder. The embeddings of the CLS token are then fed into a linear decoder and through a softmax function to get disease probability. After the model is trained, the embeddings are reduced to two-dimensional vectors, using UMAP).

### Embedding Space Interpretation Framework

The model is trained to identify disease-specific representations from each sequence to classify disease, so we expect the resulting embedding space to represent different disease stages or themes. To demonstrate this, we reduce the normalised embeddings generated by the transformer-based encoder (trained on the disease classification task) for each sequence to two-dimensional vectors U^{(p,s)}=(u₁^{(p,s)}, u₂^{(p,s)}), using the Uniform Manifold Approximation and Projection (UMAP) algorithm (McInnes et al., 2018) (e.g. see FIG. 20) To evaluate separation of disease stages in the embedding space, we examined the correlation between the reduced embeddings U and other available clinical markers F=(f₁, . . . , fₖ). We included clinically-relevant markers extracted from snapshots of EHR data such as laboratory tests, medication prescription, other co-occurring conditions (comorbidities), etc. Specifically, we computed the point-biserial correlation coefficient between each patient's reduced embeddings U^{(p,s)} and their co-occurring conditions (comorbidities), and medication prescription. We calculate the L2 norm (Euclidean distance to the origin) for each clinical marker fₖ as

$${d}_{fk} = \sqrt{{r}_{{f}_{k , {u}_{2}}}^{2} + {r}_{{f}_{k , {u}_{1}}}^{2}} ,$$

0 being no correlation between fₖ and (u₁, u₂). We then evaluated whether the most correlated conditions and medications are disease specific, and whether we find different clinical themes (e.g. see FIG. 21 which illustrates correlation between the reduced embeddings and clinical markers for each snapshot sₜ, using the Point-biserial correlation coefficient r_{f}_{ₖ}_{, u}_{ᵢ}, and calculating the distance, d_{fk}, to 0.).

### Patient Clustering

To show that each patient moves from one stage to another through time, we use the reduced embeddings per snapshot to cluster patients based on disease progression patterns. We exclude patients with less than three snapshots, and align patients' snapshots using linear interpolation, with a step chosen based on the use-case. We cluster snapshots using the k-means algorithm with multivariate dynamic time warping (DTW) (e.g. see FIG. 22). We use the embedding interpretation framework proposed in the previous section to clinically characterise and evaluate each patient cluster.

FIG. 22 is a diagram of patient clustering on trajectories (with an example 5 year time step) on simulated data. We first reduce the embeddings for each snapshot using UMAP (left). We then perform time-series clustering using the k-means algorithm with multivariate dynamic time warping (DTW) (right).

### Example of Specific Implementation of Model and Results

In the below section, a specific implementation of the model and the results of its performance is disclosed.

### Defining Study Population: Type 2 Diabetes Cohort

This research was conducted using the UK Biobank (UKBB) Resource, a large-scale research study of around 500,000 individuals (Sudlow et al., 2015), which includes primary (general practice, GP) and secondary care data (hospital) EHR data. We restrict the dataset to those that have entries in both sources, which are stored using the read and ICD ontologies (for GP and hospital respectively) (NHS). Type 2 diabetes mellitus (T2D) is one of the most prevalent chronic diseases worldwide, and patients are primarily diagnosed and managed in primary care. It presents an excellent use-case for our framework, because we have orthogonal data available to evaluate the embedding space (such as medication prescription and other co-occurring conditions). We select a cohort of 20.5 k patients with type 2 diabetes (T2D)(cases) and a corresponding cohort of 20.5 k control patients (matched on biological sex and age). Both ICD-10 and Read3 are structured in a hierarchy, so we take the parent T2D code-descriptions for hospital (ICD10) and GP (read3), and all of their children to remove all T2D associated description from all input sequences, and force the model to learn disease relevant history representations without seeing the actual diagnosis. T2D is a progressive condition, so we spliced each patient's history into three time snapshots of 10 years around diagnosis: 10 years before diagnosis, 10 years after diagnosis, and 10 to 20 years after diagnosis (e.g. see FIG. 19).

### Model Training

Using the full UKBB dataset, we first train a BertWordPiece-Tokenizer, resulting in a vocabulary size of 2025 tokens. We then train a transformer-based encoder with a hidden dimension of 200 on the Masked Language Modeling (MLM) task (Devlin et al., 2019), to learn the semantics of diagnoses. The proposed classifier uses the trained encoder and a fully connected linear layer as the decoder. To train on the classification task, we split our data set into five equally sampled folds f₀, . . . f₄ containing unique patients. We then train a total of five classification models on three folds, holding back folds fᵢ for validation and f_{(i+1)mod 5} for testing for model i, i=1, . . . , 5. All results presented are predictions and embeddings of each model on its respective independent test set. We evaluate model performance on the test set of each fold using standard metrics for binary classification, with an average recall of 0.92 and precision of 0.82 across sequences.

### Embedding Space Interpretation

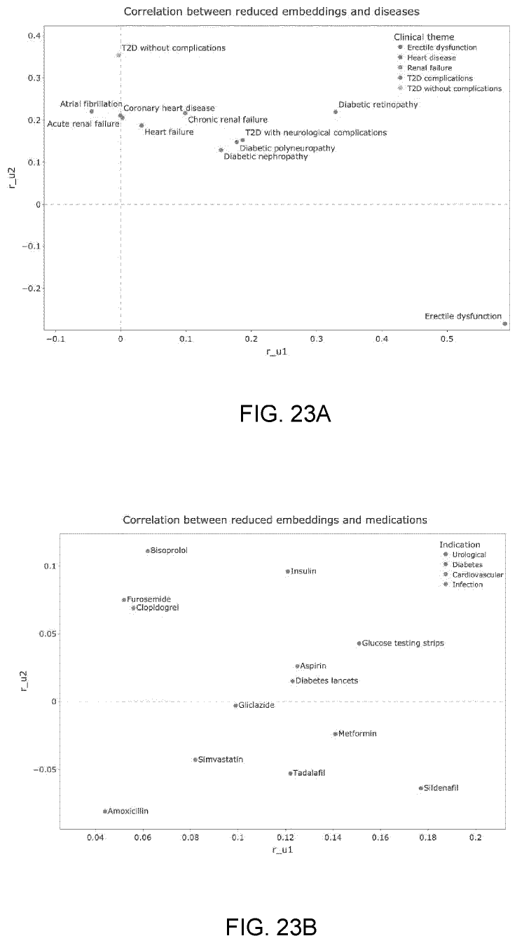

We use the default UMAP hyperparameters to reduce the embeddings to two-dimensional vectors, after experimenting with different combinations. We then look at the most strongly-correlated clinical features by taking the highest-ranked comorbid-diseases (Table 4, FIG. 23A) and medications (Table 5, FIG. 23B).

TABLE 4
Disease correlation between present comorbidities
and u1, u2, in descending order.

| Clinical theme | Disease | r_u1 | r_u2 | dist |
|---|---|---|---|---|
| Erectile dysfunction | Erectile dysfunction | 0.588 | −0.284 | 0.653 |
| Cardiovascular | Atrial fibrillation | −0.045 | 0.22 | 0.225 |
| disease | Coronary heart disease | −0.0 | 0.211 | 0.211 |
| | Heart failure | 0.032 | 0.187 | 0.19 |
| Renal failure | Chronic renal failure | 0.099 | 0.216 | 0.238 |
| | Acute renal failure | 0.003 | 0.206 | 0.206 |
| T2D complications | Diabetic retinopathy | 0.329 | 0.219 | 0.395 |
| | T2D with neurological | 0.187 | 0.152 | 0.241 |
| | complications | | | |
| | Diabetic polyneuropathy | 0.178 | 0.148 | 0.231 |
| | Diabetic nephropathy | 0.154 | 0.129 | 0.2 |
| T2D without | T2D without | −0.003 | 0.353 | 0.353 |
| complications | complications | | | |

TABLE 5
Medication correlation between present
medication prescriptions and u1, u2.

| Indication | Medication | r_u1 | r_u2 | dist |
|---|---|---|---|---|
| Cardiovascular | Aspirin | 0.125 | 0.026 | 0.127 |
| | Bisoprolol | 0.062 | 0.111 | 0.127 |
| | Simvastatin | 0.082 | −0.043 | 0.093 |
| | Furosemide | 0.052 | 0.075 | 0.092 |
| | Clopidogrel | 0.056 | 0.069 | 0.088 |
| Diabetes | Glucose testing strips | 0.151 | 0.043 | 0.157 |
| | Insulin | 0.121 | 0.096 | 0.154 |
| | Metformin | 0.141 | −0.024 | 0.143 |
| | Diabetes lancets | 0.123 | 0.015 | 0.124 |
| | Gliclazide | 0.099 | −0.003 | 0.1 |
| Infection | Amoxicillin | 0.044 | −0.081 | 0.092 |
| Urological | Sildenafil | 0.177 | −0.064 | 0.188 |
| | Tadalafil | 0.122 | −0.053 | 0.133 |

FIGS. 23A and 23B illustrates associations between reduced embeddings and clinical factors. In particular FIG. 23A illustrates association with diseases, where colours indicate broad disease theme, and FIG. 23B illustrates association with medication, where colours indicate broad indication disease theme.

We find that the diseases are either T2D complications or known comorbidities (Zghebi et al., 2020; Pearson-Stuttard et al., 2022), and medications are consistent with each disease area indication. We find three clear clinical themes:
T2D complications (positive association with both u₁ and u₂): Even though all T2D related codes were excluded in the input data, the model has learned to separate T2D without complications and T2D with complications, such as diabetic retinopathy, nephropathy, or polyneuropathy (Cheung et al., 2010). When looking at medications, we find insulin as the strongest association, which is given to severe T2D patients (Medscape, b). Moreover, T2D is a leading cause of chronic renal failure, which is found in the same area.
Erectile dysfunction (ED) (positive association with u₁): It is a prevalent comorbidity found in male T2D patients (MacDonald & Burnett, 2021), and we find tadalafil (Cialis) and sildenafil (Viagra) associated, which are used to manage ED (Medscape, c).
Cardiovascular disease (CVD) (positive association with u₂): T2D have a considerably higher risk of cardiovascular morbidity and mortality, due to high blood sugar levels causing blood vessel damage and in-creasing the risk of atherosclerosis (Einarson et al., 2018). Moreover, CVD is also driven by hypercholesterolemia, which is strongly associated with T2D. When looking at medication, we find furosemide, and bisoprolol, which are used to manage heart failure (HF) (Medscape, d), and platelet aggregation inhibitors, such as clopidogrel or aspirin, given to patients with coronary heart disease (CHD) (Medscape, a).

### Patient Clusters Evaluation

To align patients' snapshots, we use linear interpolation with a five year step, resulting in the following time steps, relative to the date of diagnosis: [−5, 0, 5, 10, 15]. We experimented with different numbers of clusters k for patient subtyping, choosing k=4. When looking at patient progression across the embedding space (e.g. see FIG. 22), we see that patients start in the same space (healthy, before diagnosis stage), and move towards disease themes or spaces, corresponding to what we see in FIG. 23A.

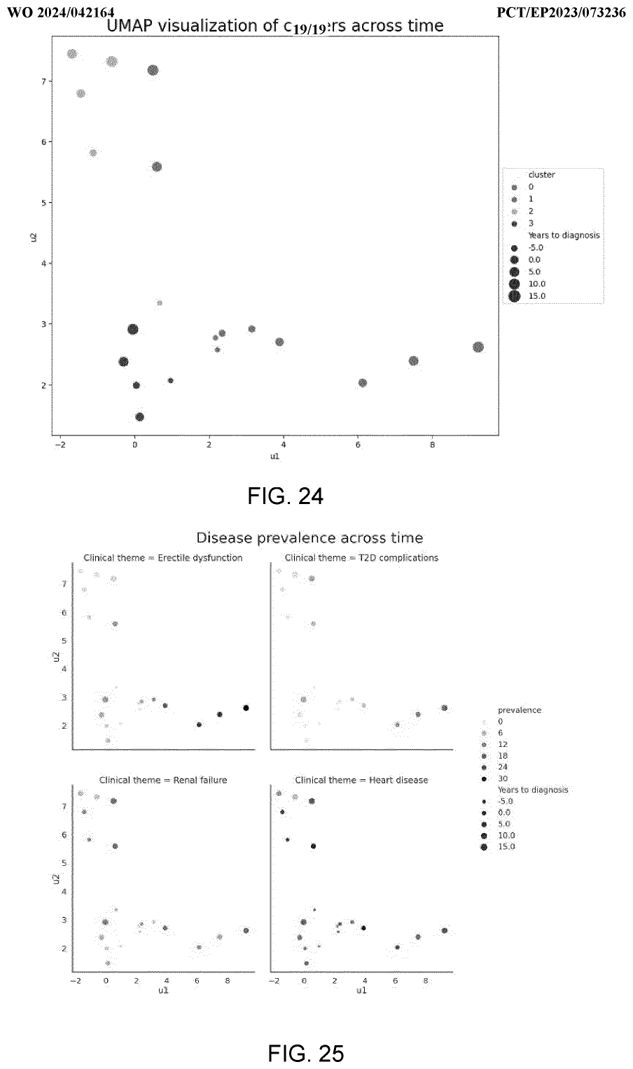

FIG. 24 is a UMAP visualisation of 4 clusters (mean per cluster and time window). Colour indicates different clusters, and size indicates time windows (the smallest is 5 years before diagnosis, and the largest is 15 years after diagnosis.)

To look at comorbidity progression, we calculate prevalence of the most strongly correlated themes, looking at how many patients had at least one diagnosis of the theme for each group and time point (FIG. 25).

Starting from the lowest u₁, u₂, we see that patients in cluster 3 stay in the well controlled state, which is also confirmed by the lack of risk factors or known comorbidities. Cluster 2 is a slightly older population that moves towards the cardiovascular and T2D without complications area. Following closely, cluster 0 represents a more severe group, with a combination of high prevalence of cardiovascular disease, renal failure and T2D complications. Finally, cluster 1 represents mostly male patients with T2D complications and erectile dysfunction.

FIG. 25 illustrates disease theme prevalence for each cluster and snapshot. Prevalence increases over time (darker colour) for each cluster.

### CONCLUSIONS

Here, we propose a framework to interpret the embedding space in a clinically meaningful way. We show that the model learns to distinguish disease-specific clinical themes, which we validate by showing associations with known T2D comorbidities and complications, and the corresponding medications. By using reduced embeddings for each time snapshot, we cluster patients and identify distinct disease progression patterns based on the clinical themes. This framework can be adapted to any disease use case, and any available clinical dataset. It can be used to both identify disease-specific information, and to identify clinically and biologically relevant groups to personalise treatment and interventions for patients.

1. A computer-implemented method of training a machine learning model to predict a clinical outcome or characteristic based on a patient's clinical history, the computer-implemented method comprising:
providing training data comprising structured electronic health record data for a plurality of patients, the structured electronic health record data comprising a plurality of clinical observations, each clinical observation having a text description and an associated time stamp, wherein the training data for each patient is labelled with one or more labels, each representing a clinical outcome or characteristic;
converting each patient's electronic health record data into a text sequence comprising the text descriptions concatenated in sequence of the associated time stamps; and
inputting the text sequence into a machine learning model and training the machine learning model to predict a clinical outcome or characteristic based on the text sequence.

2. (canceled)

3. The computer-implemented method of claim 1 wherein the computer-implemented method comprises:
masking a first percentage of words associated with the clinical outcome or characteristic from the text sequence;
randomly replacing a second percentage of words associated with the clinical outcome or characteristic from the text sequence; and
keeping a third percentage of words associated with the clinical outcome or characteristic from the text sequence.

4. The computer-implemented method of claim 3 where, when a patient's electronic health record data is labelled with multiple positive clinical outcome or characteristic labels, the computer-implemented method comprises:
generating a duplicate text sequence for each positive clinical outcome or characteristic label;
applying the steps of claim 3 for each duplicate text sequence to remove words associated with a corresponding positive clinical outcome or characteristic; and
computing, for each duplicate text sequence, loss weights for use in a loss function against which the machine learning model is trained,
wherein, for each respective duplicate text sequence, words that are associated with a positive labelled clinical outcome or characteristic that are not masked are assigned a loss weight of 0.

5. (canceled)

6. The computer-implemented method of claim 1 wherein the structured electronic health record data comprises a plurality of different electronic health record data types, each having a different ontology with different clinical codes representing the plurality of clinical observations, each clinical code having a text description, the computer-implemented method comprising:
combining the text descriptions from each data type into the text sequence in the order of their associated time stamp,
wherein the plurality of different electronic health record data types comprises one or more of:
a primary care health record, a hospital health record, a biomarker health record, a medication history record.

7. (canceled)

8. The computer-implemented method of claim 1 wherein training the machine learning model comprises a fine-tuning step and a classification training step, the fine-tuning step comprising:
masking one or more words from the text sequence, inputting the masked text sequence into the machine learning model and training the machine learning model to predict the masked words;
the classification training step comprising:
inputting the text sequence into a machine learning model and training the machine learning model to predict the clinical outcome or characteristic based on the text sequence.

9. (canceled)

10. (canceled)

11. (canceled)

12. (canceled)

13. The computer-implemented method of claim 1 wherein each clinical observation in the structured electronic health record data further comprises one or more continuous measurements, where the computer-implemented method further comprises inputting the one or more continuous measurements together with the corresponding text descriptions into the machine learning model, and training the machine learning model to predict a clinical outcome or characteristic based on the text sequence and the one or more continuous measurements,
wherein the one or more continuous measurements comprise at least one of: age of the patient, time of the clinical observation, and position of the patient.

14. (canceled)

15. The computer-implemented method of claim 13, further comprising encoding the text sequence into text embeddings, encoding the one or more continuous features into continuous feature embeddings, concatenating the text embeddings and continuous feature embeddings into an input representation, and training the machine learning model to predict the clinical outcome or characteristic based on the concatenated input representation of the text embeddings and continuous feature embeddings.

16. The computer-implemented method of claim 1 wherein the machine learning model comprises:
an encoder for mapping the text sequence to an output representation; and
a classifier layer that receives the output representation and outputs a predicted clinical outcome or characteristic,
wherein the classifier layer is trained to output a prediction of a clinical outcome or characteristic, where the prediction comprises a probability of the patient having that clinical outcome or characteristic.

17. (canceled)

18. (canceled)

19. (canceled)

20. A computer-implemented method of predicting a clinical outcome or characteristic based on a patient's clinical history, the computer-implemented method comprising:
obtaining structured electronic health record data for the patient, the structured electronic health record data comprising a plurality of clinical observations, each clinical observation having a text description and an associated time stamp;
converting the patient's electronic health record data into a text sequence by concatenating the text descriptions in sequence of the associated time stamps;
inputting the text sequence into a machine learning model trained to predict a clinical outcome or characteristic based on the text sequence; and
outputting the clinical outcome or characteristic,
wherein the clinical outcome or characteristic comprises: a phenotype, a disease diagnosis, a medical condition, a clinical outcome, a medical event, or a medical state.

21. (canceled)

22. The computer-implemented method of claim 20 wherein the machine learning model is trained to provide a prediction for a plurality of clinical outcomes or characteristics, the computer-implemented method comprising outputting the plurality of clinical outcomes or characteristics,
wherein the machine learning model is configured to provide a probability of the patient having the clinical outcome or characteristic for each of the plurality of clinical outcomes or characteristics.

23. (canceled)

24. (canceled)

25. A computer-implemented method, comprising:
obtaining structured electronic health record data for a plurality of patients which have all received a diagnosis for a particular disease, wherein the structured electronic health record data for each patient comprises a plurality of clinical observations, each clinical observation having a text description and an associated time stamp;
dividing each patient's structured electronic health record data into a plurality of datasets, wherein each dataset comprises a sequential set of clinical observations;
converting each dataset into a respective text sequence by concatenating the text descriptions of each dataset in sequence of the associated time stamps;
inputting each text sequence into an encoder of a machine learning model, wherein the encoder has been trained to map the text sequences to embeddings which encode the semantics of the text sequences;
mapping each text sequence to a respective set of embeddings using the encoder; and
performing dimensionality reduction on each set of embeddings to transform each set of embeddings into a respective reduced dimensionality embedding.

26. (canceled)

27. The computer-implemented method of claim 25, further comprising evaluating progression patterns of the particular disease based on the reduced dimensionality embeddings.

28. The computer-implemented method of claim 25, wherein all clinical observations associated with the particular disease have been deleted from each of the plurality of datasets.

29. The computer-implemented method of claim 25, wherein each of the plurality of datasets comprise clinical observations corresponding to diseases diagnoses, wherein clinical observations other than disease diagnoses have been deleted from each of the plurality of datasets.

30. (canceled)

31. The computer-implemented method of claim 25, further comprising:
performing tokenisation on each text sequence to form a sequence of word-piece tokens representing the text sequence; and inserting each sequence of word-piece tokens into the encoder.

32. The computer-implemented method of claim 25, further comprising:
computing measures of association between the reduced dimensionality embeddings and clinical factors derived from the structured electronic health record data of the plurality of patients, wherein the clinical factors derived from the structured electronic health record data of the plurality of patients comprise one or more of: symptoms, laboratory tests, vital signs, medication, and medical conditions co occurring with the particular disease.

33. (canceled)

34. The computer-implemented method of claim 32, wherein the reduced dimensionality embeddings are two-dimensional vectors each comprising a first component and a second component,
wherein computing the measures of association between the reduced dimensionality embeddings and the clinical factors derived from the structured electronic health record data of the plurality of patients comprises:
for each clinical factor, calculating a first point-biserial coefficient between the clinical factor and the first components of the two-dimensional vectors; and
for each clinical factor, calculating a second point-biserial coefficient between the clinical factor and the second components of the two-dimensional vectors,
wherein the measures of association comprises the first and second point-biserial coefficients.

35. (canceled)

36. The computer-implemented method of claim 34, further comprising:
for each clinical factor, calculating a Euclidean norm based on the corresponding first point-biserial coefficient and second point-biserial coefficient,
wherein the measures of association comprise the Euclidean norms.

37. (canceled)

38. The computer-implemented method of claim 25, wherein each dataset for each patient is associated with a respective time period defined with respect to the patient's date of diagnosis for the particular disease, and wherein the method further comprises:
performing linear interpolation on the reduced dimensionality embeddings to generate interpolated reduced dimensionality embeddings which are temporally aligned between patients; and
performing time series clustering on the interpolated reduced dimensionality embeddings to identify a plurality of patient subtypes.

39. A computer program product comprising instructions which, when executed by a computer, cause the computer to carry out the computer-implemented method of claim 1.

40. (canceled)
